(19) United States
(12) Patent Application Publication
(10) Pub. No.: US 2014/0255371 A1
(43) Pub. Date: Sep. 11, 2014

(54) Physical Optimization Beverage

(71) Applicants: Suzannah Jane ROBERTS, Bellevue, WA (US); Stephanie Jo BLACKWELL, Kirkland, WA (US); Shannon Joe BROWN, Grass Valley, CA (US)

(72) Inventors: Suzannah Jane ROBERTS, Bellevue, WA (US); Stephanie Jo BLACKWELL, Kirkland, WA (US); Shannon Joe BROWN, Grass Valley, CA (US)

(73) Assignee: COGNATE3 LLC, Bellevue, WA (US)

(21) Appl. No.: 14/201,865

(22) Filed: Mar. 9, 2014

Related U.S. Application Data
(60) Provisional application No. 61/774,626, filed on Mar. 8, 2013.

(51) Int. Cl. A23L 2/52; A23L 1/29; A61K 31/122; A61K 45/06

(52) U.S. Cl. CPC A23L 2/52; A61K 45/06; A23L 1/293; A61K 31/122
USPC 424/94.1; 426/231; 426/2; 426/590; 426/72; 426/73; 426/597; 426/593; 426/548; 426/74

(57) ABSTRACT

The present invention provides novel methods of making a non-corrosive base solution for use as an alkalinity increasing agent and/or antioxidant. The compositions and methods described herein are useful for optimizing health and performance; preventing illness; decreasing recovery times from exertion, illness, and injury; increasing energy levels; improving exercise performance; improving hydration; preventing muscle damage after exercise; and increasing stamina during exercise. The present invention further provides novel compositions and methods which can be used to provide relief from disorders related to or complicated by acidosis or excessive free radical or other reactive oxygen species production.

### RELATED APPLICATIONS

This application claims priority benefit of U.S. Provisional patent application Ser. No. 61/774,622, tiled Mar. 8, 2013 and U.S. Provisional patent application Ser. No. 61/774,626, filed Mar. 8, 2013, the disclosure of each which is incorporated herein in its entirety by reference.

### TECHNICAL FIELD

The present invention relates to methods of making a non-corrosive base solution and the use of the non-corrosive base solution to alter physiological pH in mammalian subjects. More specifically, the present invention relates to methods and compositions for optimizing health and performance; preventing illness; decreasing recovery times from exertion, illness, and injury; increasing energy levels; improving exercise performance; improving hydration; preventing muscle damage after exercise; and increasing stamina during exercise.

### BACKGROUND

As people exercise, heart rate, systolic blood pressure, and cardiac output increase. The body's metabolism becomes more active, producing CO₂ and H⁺, and respiration increases to compensate for increased oxygen demand. Eventually, an individual's metabolism exceeds the body's oxygen supply and the body uses alternate biochemical processes such as the lactic acid system (anaerobic glycolysis) to generate energy. These chemical changes can cause the pH of the blood to drop. (H+) accumulation, coinciding with, but not caused by lactate production, results in acidosis, impairing muscle contraction, and ultimately leading to the ‘burn’ and associated weariness (Robergs, Ghiasvand, Parker 2004).

The normal pH of the muscle cell is 7.1 but if the buildup of H+ continues and pH is reduced to around 6.5 then muscle contraction may be impaired. (Garrett, 2000) When an individual's exercise intensity is sufficient to cross the lactic threshold, i.e. there is an abrupt increase in blood lactate levels, exercise becomes more difficult. (Roberts Robergs 1997). Muscles ache and burn and become extremely fatigued. Symptoms increase if the exercise continues making it difficult if not impossible to maintain exercise levels at the desired intensity. Studies have repeatedly found high correlations between performance in endurance events such as running, cycling, and race-walking and the maximal steady-state workload at the lactate threshold (McKardle, Katch, & Katch 1996).

For optimal health, the pH of blood should be about 7.365. Higher levels of pH can lead to alkalosis (pH above ˜7.5) and lower levels of pH can lead to acidosis (<7.0). An alteration in normal physiological pH such as that experienced during intense exercise, can cause nausea, vomiting, hyperventilation, abdominal pain, lethargy, anxiety, confusion, shock, severe anemia, hypotension, irregular heart rate and tachycardia. It can also make an individual more susceptible to infection and disease by creating a more hospitable environment for microorganisms to grow.

Along with intense exercise, physiolocal pH levels can be affected by a number of exterior factors including, illness, stress, diet, exposure to toxins, and poor sleeping habits. Respiratory acidosis may be caused by chronic obstructive pulmonary disease, asthma, respiratory depressants or sleep apnea. Metabolic acidosis may be caused by ethanol, diabetic ketoacidosis, uremia, sepsis, shock, methanol/ethylene glycol, salicylate overdose, diarrhea and carbonic anhydrase inhibitors.

The human body attempts to maintain optimal pH through the actions of buffers, respiration, and renal function. In dealing with the normal acid load from diet and metabolism, buffers such as proteins, phosphate and H₂CO₃:HCO₃— act to control the level. Respiration maintains a constant carbonic acid level at 1.2 meq/l or PaCO₂ of 40 mmHG through either excretion or retention of CO₂ by the lungs. Respiration can also rapidly compensate for changes in pH by altering the level of PaCO₂ through the alteration of alveolar ventilation. The renal system manipulates the volume and composition of extracellular fluid to help maintain the pH of plasma. However, while the renal system can correct states of excess, it cannot correct states of deficiency such as through loss of Na+, K+ or HCO₃⁻. Additionally, unlike respiratory regulation, regulation of pH through renal function can take several days.

Failure or overloading of any regulatory mechanism, whether through stress, pharmacological treatments, diet, exercise, or disease can cause acidosis, which is at best uncomfortable and at worse can cause nausea, vomiting, hyperventilation, abdominal pain, lethargy, anxiety, confusion, shock, severe anemia, hypotension, irregular heart rate and tachycardia as well as leaving an individual more susceptible to infection and conditions such as cancer, cardiovascular disease, fibromyalgia, hepatic disease, gout, arthritis, anemia, weight gain, diabetes, cellulitis and pancreatic impairment. There is therefore a need for compositions that can compensate for the failure of regulatory mechanisms to maintain physiological pH and prevent acidosis regardless of the cause of the acidosis. There is an additional need for compositions that can compensate for the failure of regulatory mechanisms to maintain physiological pH despite continued insult to an individual's system.

### SUMMARY

Provided herein are compositions and methods for maintaining optimal physiological pH and treating or preventing acidosis in physiological fluids or compartments of the mammalian body, including in humans. Compositions and methods as described herein may further be used in the treatment of conditions caused or exacerbated by acidosis, including for example lactic acidosis.

Acidosis may be accompanied by the buildup of lactate, particularly D-lactate. This buildup of lactate generally occurs when cells are hypoxic and functioning anaerobically whether due to illness and disease or intense, exercise. Impaired cellular respiration leads to lower pH levels and can be indicative of tissue hypoxia, hypoperfusion, and possible damage. Compositions and methods of the present invention increase pH levels, preventing or treating lactic acidosis (lactate levels>5 mmol/L and serum pH<7.35).

Compositions and methods as described herein may treat or prevent acidosis by a variety of means. In some embodiments, the compositions and methods of the present invention may decrease lactate levels, specifically D-lactate. The normal blood lactate concentration is 0.5-1.0 mmol/L. Individuals in various disease states may have lactate concentrations of less than 2 mmol/L. Hyperlactemia is defined as a mild-to-moderate persistent increasing blood lactate concentration (2-5 mmol/L) without metabolic acidosis, whereas lactic acidosis is characterized by persistently increased blood lactate levels (usually >4-5 mmol/L) in association with metabolic acidosis. In other embodiments, the compositions and method of the present invention may increase the physiological alkalinity of a mammalian subject; bring pH levels to within normal ranges such as between 7.35 and 7.45 for blood and 4.6 and 8 for urine.

The normal pH of intracellular and interstitial fluids is maintained because acids are removed at the same rate they are added. If acid is added faster than it is removed, the pH of intracellular and interstitial fluids decreases, resulting in acidosis. While strong mineral bases have often been used to neutralize acids, they are very corrosive and are not generally suitable for altering pH in living organisms. The present invention provides compositions and methods for altering base solutions so that they may be effectively used to increase levels in living organisms.

The methods and formulations of described herein provide a base solution with a high concentration of OH⁻ ions which may be used as an alkalinity increasing agent or as an antioxidant. Such a base solution or alkaline water may be formed by any means that generates a solution with a high concentration of OH⁻ ions, specifically an oxide or hydroxide combined with purified, distilled, spring, filtered. or mineral-free water.

The alkaline water described herein may be used for a variety of purposes including preventing and treating illness, optimizing health and performance, decreasing recovery times from exertion, and increasing stamina during exercise.

Alkaline water may be manufactured by whatever means useful to create a water with a pH between about 7 to about 14, preferably a pH of about 7.5 to about 12.75, preferably a pH of about 10 to about 11, about 12 to about 14, about 12.25 to about 12.75, more preferably about 12.3 to about 13.8, preferably a pH of about 12.5 to about 13.75.

In some embodiments, alkaline water may be manufactured by combining oxides including, but not limited to, calcium hydroxide, calcium oxide, sodium hydroxide, sodium oxide, potassium hydroxide, potassium oxide, magnesium hydroxide, or magnesium oxide, with mineral free water to create a non-corrosive base solution with a high concentration of OH− ions.

The solution containing the oxide or hydroxide and water is stirred to increase a rate or amount of dissociation of the OH⁻ ions. After mixing, the ionic concentration in the water will result in a conductivity measurement of between about 50 μS/cm to about 2000 μS/cm, preferably between about 100 μS/cm to about 1000 μS/cm, preferably about 500 μS/cm to about 800 μS/cm, preferably about 650 μS/cm to about 750 μS/cm, preferably about 700 μS/cm to about 2000 μS/cm, preferably about 700 μS/cm to about 750 μS/cm.

The resulting concentrated alkaline water may be consumed directly or diluted with filtered water to a pH of about 7 to about 10, about 7.5 to about 8. The diluted alkaline water will have a conductivity of about 45 μS/cm to about 90 μS/cm, in some embodiments about 50 μS/cm to about 75 μS/cm, and in certain embodiments about 50 to about 60 μS/cm.

In some embodiments, the alkaline water may be made with calcium hydroxide. Calcium hydroxide (Ca(OH)₂) is a base which will only dissociate slightly in a weak acid environment. At a pH of 5.5 or higher, calcium hydroxide rapidly loses its solubility and at a pH of 8.0 it is insoluble. In one embodiment, a method is provided herein of increasing the solubility of calcium hydroxide allowing a larger volume of Ca(OH)₂ to be dissociated in solution including weakly acidic, neutral or slightly basic solutions. In a further embodiment., a means of raising the pH of a Ca(OH)₂ solution at least one pH point higher than a normal saturated calcium hydroxide solution is provided. In sonic embodiments, a method of increasing the reactivity of Ca(OH)₂ in solution is provided.

Useful forms of calcium hydroxide for use within the formulations and methods of the invention include the forms described herein, as well as solvates, hydrates, or combinations thereof.

Sulfuric acid is a strong mineral acid. In the compositions and methods of the present invention, sulfuric acid in water is treated to reduce the acidity while maintaining the concentration of sulfate in the solution. Such treatment may be accomplished by any means possible, including the addition of oxygen to the sulfuric acid solution. In some embodiments, the sulfuric acid solution is infused with ozone. Such treatments may increase the pH of the sulfuric acid solution, creating a neutral or basic solution which may then be combined with the calcium hydroxide solution described above to create a non-corrosive base solution with a high concentration of OH⁻ ions.

In another embodiment, calcium oxide (CaO), another strong base, is used as the acid neutralizing agent and/or antioxidant. Calcium oxide is stirred into purified, distilled or mineral free water to create a non-corrosive base solution with a high concentration of OH⁻ ions. Such a solution will have a pH between about 7 to about 14, preferably a pH of about 12 to about 14, more preferably about 12.3 to about 13.8, preferably a pH of about 12.5 to about 13.75 and an ionic concentration such that the conductivity would be about 50 μS/cm to about 2000 μS/cm, preferably between about 100 μS/cm to about 1000 μS/cm, preferably about 500 μS/cm to about 800 μS/cm. preferably about 650 μS/cm to about 750 μS/cm, preferably about 700 μS/cm to about 750 μS/cm. In some embodiments the ionic concentration is such that the conductivity may be between about 700 μS/cm to about 200 μS/cm.

The resulting concentrated alkaline water may be consumed directly or diluted with filtered water to a pH of about 7 to about 10, about 7.5 to about 8. The diluted alkaline water may have a conductivity of about 45 μS/cm to about 90 μS/cm, about 50 μS/cm to about 75 μS/cm, about 50 μS/cm to about 60 μS/cm.

In exemplary embodiments, the compositions and methods described herein employ a base solution (also referred to as an OH⁻ solution or alkaline water) as described above to increase or maintain physiological pH in a mammalian subject.

Alkaline water as described herein may be used to increase physiological pH in a mammalian subject for a variety of reasons including to speed recovery times, particularly from periods of stress or intense physical activity; to increase endurance; to increase physical performance; to optimize health and performance; and to prevent illness.

Alkaline water as described herein may additionally be used to treat symptoms of acidosis in mammalian subjects. Such symptoms include, but are not limited to, extreme tenderness in the joint, inflammation, swelling, pain, redness in the affected area, confusion, lethargy, rapid breathing, shortness of breath, wheezing, chest pain or pressure, joint stiffness, swelling, joint deformity, crepitus, non-specific fever, joint inflammation, headaches, fatigue, constipation, a feeling of euphoria, nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, easy bruising, vomiting, ascites, dry skin, dry mouth, low blood pressure, frequent urination, chest pain, lymph node pain, night sweats, skin rash, hyperventilation, abdominal pain, severe anemia, muscoskeletal pain, memory issues, and light headedness.

Alkaline water as described herein may additionally be used to treat mammalian subjects with acidosis, as well as conditions associated with or complicated by acidosis including, but not limited to, methicillin resistant staphylococcus aureus (MRSA), sepsis, folliculitis, gout, arthritis, hypoxia, hypoperfusion, hemorrhage, ethanol toxicity, shock, hepatic disease, diabetic ketoacidosis, exercise fatigue, non-Hodgkin's and Burkitt's lymphoma, hyperventilation, abdominal pain, lethargy, shock, severe anemia, hypotension, irregular heart rhythm, tachycardia, fibromyalgia, weight gain, cancer, cardiovascular disease, respiratory disease, infection, diabetes, cellulitis and pancreatic impairment, in other embodiments, the compositions and methods of the present invention are used as anti-bacterial agents.

These and other subjects are effectively treated prophylactically and/or therapeutically by administering to the subject an effective amount of alkaline water.

In some embodiments, alkaline water may be taken in a concentrated formulation with a pH of between about 12 to about 13.75, preferably about 12 to about 12.5 and a conductivity of about 700 μS/cm to about 200 μS/cm, preferably about 700 μS/cm to about 1500 μS/cm, preferably about 700 μS/cm to about 1000 μS/cm, more preferably about 700 μS/cm to about 750 μS/cm. In other embodiments, Alkaline water as described herein may be diluted with water to a pH of about 6.9 to about 7.5, in some embodiments about 6.9 to about 7.2, and in other embodiments about 7.0, with a conductivity 45 μS/cm to about 60 μS/cm, and in some embodiments about 50 μS/cm to about 55 μS/cm. Any type of water that will lower the pH may be used for the dilution including, but not limited to, tap, spring, distilled, reverse osmosis, mineral-free or filtered water.

Alkaline water may be taken alone, or in a coordinate or combined formulation with one or more additional agents to optimize health and performance; prevent illness; decrease recovery times from exertion, illness and injury; and extend endurance during exercise.

Useful secondary or additional agents for use within the formulations and methods of the present invention include, but are not limited to, alkalinity increasing agents, adaptogens, amino acids and amino acid derivatives, anti-inflammatory agents, anti-nausea agents, analgesics, antioxidants, aphrodisiacs, detoxifying agents, dietary supplements, herbal supplements, calming agents, herbs and plant extracts, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein.

The compositions described herein may additionally contain sweeteners, stabilizers, flavoring, anti-caking agents, flavor protectants, preservatives, anti-foaming agents, colorants, emulsifiers and the like.

The foregoing and other objects, features, aspects and advantages of the present invention will become more apparent from the following detailed description of the invention and examples, which are intended to exemplify non-limiting embodiments of the invention.

### DETAILED DESCRIPTION

Provided herein are novel methods and compositions for maintaining optimal pH in mammalian subjects and treating or preventing acidosis, symptoms of acidosis, and conditions caused by or exacerbated by acidosis. Such methods and compositions as described herein are effective for optimizing health and performance; preventing illness; decreasing recovery times from exertion, illness, and injury; increasing energy levels; improving exercise performance; improving hydration; preventing muscle damage after exercise; and increasing stamina during exercise.

Formulations and methods described herein provide a non-corrosive strong base solution (also referred to as an OH⁻ solution, a base solution, or alkaline water) and methods for using the solution for the regulation of physiological pH in vertebrates, including mammals.

The formulations described herein may be manufactured and sold in a variety of forms. In some embodiments, they may be manufactured and sold as a single strength beverage for direct consumption by the consumer. In other embodiments, the formulations may be sold in an aqueous concentrate to be diluted with water to yield a beverage that treats or prevents acidosis, symptoms of acidosis, and conditions caused by or exacerbated by acidosis. The formulations may also be sold as a powder, granule formation, or tablet which is to be dissolved in water to yield a beverage that treats or prevents acidosis, symptoms of acidosis, and conditions caused or exacerbated by acidosis.

Mammalian subjects amenable for treatment according to the formulations and methods of the invention further include, but are not limited to, human and other mammalian subjects at risk for or with symptoms of acidosis, as well as symptoms or conditions associated with or complicated by acidosis. Acidosis may be caused by any of a variety of reasons including, but not limited to, intense exercise, illness, stress, diet, exposure to toxins, and poor sleeping habits.

Compositions and methods described herein are useful for optimizing health and performance; preventing illness; decreasing recovery times from exertion, illness, and injury; increasing energy levels; improving exercise performance; improving hydration; preventing muscle damage after exercise; and increasing stamina during exercise. They are additionally useful for treating, preventing or alleviating symptoms of acidosis, including, but not limited to, extreme tenderness in the joint, inflammation, swelling, pain, redness in the affected area, confusion, lethargy, rapid breathing, shortness of breath, wheezing, chest pain or pressure, joint stiffness, swelling, joint deformity, crepitus, non-specific fever, joint inflammation, headaches, fatigue, a feeling of euphoria and nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, easy bruising, vomiting, ascites, easy bruising, dry skin, dry mouth, low blood pressure, frequent urination, chest pain, lymph node pain, night sweats, skin rash, hyperventilation, constipation, severe anemia, memory loss, mood swings, musculoskeletal pain and light headedness.

The formulations and methods described herein are additionally useful in the treatment of mammalian subjects with acidosis, as well as conditions associated with or complicated by acidosis including gout, abdominal pain, Alzheimer's disease, amyotrophic lateral sclerosis, fungal infections including but not limited to candidiasis, arthritis, atherosclerosis, cancer, cardiovascular disease, cataracts, cellulitis and pancreatic impairment, chronic obstructive pulmonary disease, coronary artery disease, diabetes, diabetic ketoacidosis, ethanol toxicity, exercise fatigue, Folliculitis, gout, heart failure, hemochromatosis, hemorrhage, hepatic disease, hepatitis C, hypertension, hyperventilation, hypotension, hypoxia and hypoperfusion, infection, inflammatory bowel disease, irregular heart rhythm, Lesch-Nyhan syndrome, lethargy, macular degeneration, methicillin resistant staphylococcus aureus (MRSA), Morgellons disease, fibromyalgia, multiple sclerosis, nausea, non-Hodgkin's and Burkitt's lymphoma, Parkinson's disease, psoriasis, regional hypoperfusion, pancreatic impairment, reperfusion injury, respiratory disease, Reynaud's phenomenon, sepsis, severe anemia, shock, tachycardia, tinea cruris, tinea pedis, candidiasis, vomiting and weight gain.

A further embodiment of the present invention provides a strong base solution (also referred to as an OH⁻ solution, a base solution or alkaline water) for use in the prevention of secondary infections in vertebrates, including mammalian subjects; particularly mammalian subjects with compromised immune systems, such as those subjects suffering from chronic diseases such as, but not limited to, cancer or HIV, or whose immune systems are compromised due to treatments for diseases such as cancer.

Formulations and methods described herein further provide methods of using the non-corrosive strong base solution (also referred to as an OH⁻ solution, a base solution or alkaline water) as an antioxidant as Further described in related (US CON XX, U.S. patent application Ser. No. 12/167,123 filed Jul. 2, 2008 which claims priority benefit of U.S. Provisional Patent Application No. 60/967,633 filed Jul. 2, 2007.

Antioxidants may be used in the reduction of reactive oxygen species in vertebrates, including mammals. Reduction of free radicals and other reactive oxygen species (ROS) is effective in the treatment of diseases including, but not limited to, gout, abdominal pain, Alzheimer's disease, amyotrophic lateral sclerosis. arthritis, atherosclerosis, cancer, cardiovascular disease, cataracts, cellulitis and pancreatic impairment, chronic obstructive pulmonary disease, coronary artery disease, diabetes, diabetic ketoacidosis, ethanol toxicity, exercise fatigue, folliculitis, gout, heart failure, hemochromatosis, hemorrhage, hepatic disease, hepatitis C, hypertension, hyperventilation, hypotension, hypoxia, hypoperfusion, infection, inflammatory bowel disease, irregular heart rhythm, Lesch-Nyhan syndrome, lethargy, macular degeneration, methicillin resistant staphylococcus aureus (MRSA) fibromyalgia, multiple sclerosis, nausea, non-Hodgkin's and Burkitt's lymphoma, Parkinson's disease, psoriasis, regional hypoperfusion, pancreatic impairment, reperfusion injury, respiratory disease, Reynaud's phenomenon, sepsis, severe anemia, shock, tachycardia, vomiting and weight gain.

Formulations and methods herein may additionally employ a base solution as an antioxidant or free radical scavenger for the regulation of ROS levels including free radical levels. Within these formulations and methods, the calcium hydroxide or calcium oxide or other oxides and hydroxides used to produce the OH⁻ solution may be provided in any of a variety of forms, including solvates, hydrates, or combinations thereof. Formulations containing a non-corrosive strong base solution made from calcium hydroxide or calcium oxide or other hydroxide or oxide as disclosed herein are effectively used to treat mammalian subjects suffering from an over accumulation of free radicals as well as diseases and conditions associated with free radicals.

For the purposes of describing the present invention, the following terms and definitions are provided by way of example. Additional terms and definitions for describing embodiments of the present invention are provided by way of example elsewhere in the application.

As used herein, “microbial” refers to any microorganism capable of causing disease. Such microorganisms include fungal, viral and bacterial microorganisms.

By the term “effective amount” of a compound is meant a non-toxic but sufficient amount of the compound to provide the desired function, i.e., anti-infective, as an antioxidant, or acid-neutralizing agent. An appropriate effective amount may be determined by one of ordinary skill in the art using only routine experimentation.

Formulations and methods herein employ alkaline water or an OH⁻ solution alone or with one or more additional agents to optimize health and performance; prevent illness; decrease recovery times from exertion, illness and injury; and extend endurance during exercise. Useful secondary or additional agents for use within the formulations and methods of the present invention include, but are not limited to., alkalinity increasing agents, adaptogens, amino acids and amino acid derivatives., anti-inflammatory agents, anti-nausea agents, analgesics, antioxidants, aphrodisiacs, detoxifying agents, dietary supplements, herbal supplements, calming agents, herbs and plant extracts, flavorings, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein. Within these formulations and methods, the secondary agent may be provided in any of a variety of forms, including any polymorphs, enantiomers, pharmaceutically acceptable salts, solvates, hydrates, or combinations thereof. Such combinations of an OH⁻ composition and secondary agent may be administered either combinatorially or coordinately as disclosed herein to effectively optimize health and performance; prevent illness; decrease recovery times from exertion, illness, and injury; increase energy levels; improve exercise performance; improve hydration; prevent muscle damage after exercise; and increase stamina during exercise.

Alkalinity increasing agents for use within the formulations and methods of the present invention include, but are not limited to sodium bicarbonate; a carbonate, a phosphate, or a hydroxide of sodium or potassium; magnesium carbonate; magnesium hydroxide; ammonium carbonate; ammonium bicarbonate; magnesium oxide; sodium or potassium citrate, bicarbonate, sulfate, and benzoate; ascorbate; calcium carbonate; any pharmaceutically acceptable material that causes the pH of an aqueous medium to rise above pH 7.0, or mixtures thereof.

Formulations and methods as described herein may include adaptogens. An adaptogen is a metabolic regulator which increases the ability of an organism to adapt to environmental factors, and prevents damage from such factors. Exemplary adaptogens include, but are not limited to, ashwagandha, eleutherococcus senticosus, reishi, astragalus, licorice root, panax quinquefolius, panax ginseng and schisandra berries.

Antioxidants included in the formulations provided herein may be in the form of nutritional supplements such as, but not limited to, vitamin A, vitamin C, vitamin E, erythorbic acid, beta-carotene, carotenes, lutein, manganese, lycopene, melatonin, or coenzyme Q10 or may be present in plant extracts, either of which may be combined with the alkaline water. Plant extracts containing antioxidants may come from plant sources such as, but not limited to, apricot, acai fruit, acerola, apple, blueberry, blackberry, black currant, carrots, cherry, chokeberry, cranberry, elderberry, green tea, goji berry, grape seed, mangosteen, maqui berry, milk thistle, pomegranate seed, prune, raspberry, red grape, rooibos, rosehips, strawberry, seabuckthorn, while grape, whole grape, yumberry and acerola fruit.

Formulations and methods herein may further employ vitamin, mineral and nutritional supplements in a variety of forms including, but not limited to, vitamin B complex, folic acid, niacin, niacinamide, pantothenic acid, pyridoxine HCl, vitamin B2, folate, biotin, vitamin. C, vitamin D, vitamin D₃, vitamin E, vitamin K, cyanocobalamin, inositol, thiamine, thiamine mononitrate, calcium pantothenate, mixed tocophyerols, d-alpha tocopheryl acetate, magnesium, calcium, calcium carbonate, calcium chelate, calcium di-phosphate, calcium phosphate, iron, magnesium carbonate, magnesium citrate, magnesium oxide, magnesium phosphate, manganese chelate, manganese sulfate, potassium, potassium chelate, potassium chloride, sodium, zinc, vanadyl sulphate, chromium, chromium chloride, chromium picolinate, and chromium polynicotinate. The amount of vitamin, mineral and nutritional supplements may vary in the formulations and methods described herein. In some embodiments, the formulations may comprise independently between about 1% and to about 250% of the U.S. recommended daily allowance, about 10% to about 250%, about 10% and 150%, between about 10% to about 100%, about 10% to about 50% of the U.S. recommended daily allowance of any one vitamin, mineral or nutritional supplement. In some embodiments the composition as described herein has between 10% to 250%, about 10% to about 150%, about 25% to about 100%, about 50% to about 75% of the U.S. recommended daily amount of vitamin B₃ or vitamin B complex.

In some embodiments, the compositions and methods described herein may further include amino acids, amino acid precursors, and amino acid derivatives whether branched or straight chain amino acids. Exemplary amino acids, precursors and derivatives which may be used in the formulations and methods described herein include, but are not limited to, 5-HTP, arginine, beta alanine, carnitine fumarate, citrulline malate, glutamine peptide, glycine, 1-alanine, 1-arginine, 1-arginine hydrochloride, 1-histidine, 1-methionine, 1-lysine HCL, 1-phenylalanine, leucine ethyl ester, 1-glutamine, 1-isoleucine, 1-theanine, 1-tyrosine, phenylalanine, taurine, tri-methyl glycine, tryptophan, tyrosine, 1-carnitine., 1-carnosine, glutamine alpha ketoglutarate and alpha-L-polylactate.

During exercise and illness, dehydration and electrolyte balance can be an issue leading to muscle cramps, loss of appetite, dizziness, irregular heartbeat, muscle weakness, confusion, nausea, and muscle spasms. The compositions and methods described herein may therefore include electrolytes to maintain homeostasis. Electrolytes used herein include, but are not limited to, sodium chloride, sodium acetate, acidic sodium citrate, acidic sodium phosphate, sodium chloride, sodium bicarbonate, sodium bromide, sodium citrate, sodium lactate, sodium molybdate, sodium phosphate, anhydrous sodium sulphate, sodium sulphate, sodium tartrate, sodium benzoate, sodium selenite, and other sodium salts and mixtures thereof; potassium chloride, potassium acetate, potassium bicarbonate, potassium bromide, potassium citrate, potassium-D-gluconate, monobasic potassium phosphate, potassium tartrate, potassium sorbate, potassium iodide, and other potassium salts and mixtures thereof; magnesium carbonate, magnesium citrate, magnesium oxide, magnesium phosphate, as well as other magnesium salts and mixtures thereof; calcium chloride, calcium carbonate, calcium chelate, calcium di-phosphate, calcium lactate, calcium phosphate tribasic and other calcium salts and mixtures thereof. Such electrolytes may be included in the formulations described herein in proportions and amounts suitable to replenish salts lost during exercise or illness or otherwise depleted.

Additional agents which may be used in the compositions and methods described herein include anti-inflammatory agents including, but not limited to, extracts from plants such as maqui berry, milk thistle, skull cap, red raspberry, red sour cherry, green tea and hops.

Other agents which may be used in the compositions and methods described herein include anti-nausea agents including, but not limited to, extracts from peppermint, ginger and chamomile.

A further agent which may be used in the compositions and methods described herein includes analgesic agents such as, but not limited to, white willow bark.

The formulations and methods described herein may additionally include herbal supplements and extracts with beneficial properties including, but not limited to, passion flower, horny goat weed, skullcap, milk thistle, Echinacea, dandelion leaf, St. John's wort, green tea, black tea, chamomile or peppermint, or an extract thereof.

The formulations and methods described herein may further include plants with beneficial properties including, but not limited to, guarana seeds, acerola berries, coconut water, yerba mate, acai berry, ginseng root, panax ginseng root, ginkgo biloba, white willow bark, acacia, ashwagandha, chokeberry, elderberry, cranberry, maqui berry, blueberry, pomegranate, rooibos, goji berry, elder berry, valerian, seabuckthom, yumberry, blackberry, astragalus, damiana, and ginger.

Mammalian subjects suffering from acidosis may be exercising intensely. It is important to maintain energy levels to continue the exercise. The compositions and methods described herein may further include energy boosters that may increase performance including, but not limited to, creatine ethyl ester, creatine monohydrate, magnesium creatine chelate, creatine hydrochloride, creatine nitrate, creatine monohydrate and royal jelly.

Flavonoids are plant pigments present in a wide range of fruits, vegetables, and nuts. In humans, they may have anti-inflammatory, anti-cancer and anti-viral properties. Useful flavonoids within the compositions and methods of the present invention are present in chamomile extract, cocoa powder, red grape, black tea, and white tea, ginkgo biloba, berries, parsley, and green tea some or all of which may be included in the compositions and methods described herein.

The Alkaline water compositions described herein may additionally include sedatives to encourage relaxation or to make those suffering from illness and disease more comfortable. Such sedatives include, but are not limited to, including, but not limited to, lavender, lemon balm, lemongrass, linden, oatstraw, St. John's wart, valerian root, kava kava, hops and passion flower.

The formulations and methods described herein may further include stimulants. Such stimulants include, but are not limited to, caffeine, citicoline, d-glucuronolactone, guarana extract, ginseng, concentrated green tea, green coffee beans, glucuronolactone, guarana, panax ginseng, panax quinquefolius, Siberian ginseng, and theobromine.

Additional agents which may be included in the formulations and methods described herein are immune boosters including, but not limited to, Echinacea and astragalus root.

The compositions herein may additionally comprise one or more flavoring agents. Such flavorings are any flavoring typically included in a beverage composition including, but not limited to synthetic flavorings, fruit juice, vegetable juice, milk solids, fruit flavors, herbal flavor and mixtures thereof. The fruit juice can be any citrus juice, non-citrus juice, or mixture thereof, which is known for use in dilute juice beverages. The juice can be extracted from, but riot limited to, apple, cranberry, pear, peach, plum, apricot, nectarine, grape, guava, cherry, currant, raspberry, gooseberry, elderberry, blackberry, blueberry, strawberry, lemon, lime, mandarin, orange, tomato, lettuce, dandelion, rhubarb, pineapple, coconut, pomegranate, kiwi, mango, papaya, banana, watermelon, passion fruit, tangerine, and cantaloupe. The vegetable juice can be any vegetable juice generally consumed including, but not limited to, celery, spinach, cabbage, watercress, carrot, beet, spirulina, sweet potato, kale, romaine, collard greens, endive, escarole, bok choy, fennel, parsley, wheat grass, or cucumber. Such fruit and vegetable juices may or may not have additional beneficial properties such as antioxidants and/or flavonoids.

Formulations and methods herein may additionally include a protein source. Protein sources include, but are not limited to, milk solids, calcium caseinate, whey protein concentrate, whey protein isolate, whey protein hydrolysate, soy protein, casein hydrolysate, rice protein, wheat protein, corn protein, partially hydrolyzed whey protein, or ultra-filtered whey protein.

Formulations and methods herein may further include one or more sweeteners or carbohydrate source. Sweeteners which may be used in the formulations herein include, but are not limited to, acesulfame potassium, aspartame, cane sugar, beet sugar, corn syrup, crystalline fructose, dextrose, D-ribose, fructose, glucose, glucose-fructose syrup, high fructose corn syrup, high fructose liquid sugar, honey, maltodextrin, sorbitol, stevia, sucralose, sucrose, sugar, trehalose, truvia or xylitol.

A broad range of mammalian subjects, including human subjects, are amenable for treatment using the formulations and methods of the invention. The compositions and methods described herein are particularly useful for optimizing health and performance; preventing illness; decreasing recovery times from exertion, illness, and injury; increasing energy levels; improving exercise performance; improving hydration; preventing muscle damage after exercise; and increasing stamina during exercise. The formulations described herein may be used prophylactically or therapeutically. In some embodiments, the formulations described herein may be administered to a mammalian subject prior to or during strenuous exercise in order to prevent or reduce acidosis or symptoms or sequelae of acidosis. In some embodiments, the formulations described herein may be used to treat mammalian subjects with acidosis or symptoms of acidosis including, but not limited to, extreme tenderness in the joint, inflammation, swelling, pain, redness in the affected area, confusion, lethargy, rapid breathing, shortness of breath, wheezing. chest pain or pressure, joint stiffness, swelling, joint deformity, crepitus, non-specific fever, joint inflammation, headaches, fatigue, a feeling of euphoria and nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, easy bruising, vomiting, ascites, easy bruising, dry skin, dry mouth, low blood pressure, frequent urination, chest pain, lymph node pain, night sweats, skin rash, hyperventilation, constipation, severe anemia, memory loss, mood swings, musculoskeletal pain and light headedness.

In other embodiments, the formulations described herein may be used to treat human and other mammalian subjects with acidosis and/or excessive free radical production as well as those suffering from conditions or complications of having acidosis including increased susceptibility to microbial infections or other secondary infections; skin infections such as, but not limited to, psoriasis, Morgellons disease, and fungal infections such as candidiasis, tinea cruris, and tinea pedis; cancer; diabetes; cellulitis: or pancreatic impairment; and/or mammals in need of antioxidant treatment or free radical elimination, including those suffering from conditions or complications associated with excess free radicals, including, but not limited to, gout. Lesch-Nyhan syndrome, hemochromatosis, Alzheimer's disease, amyotrophic lateral sclerosis, arthritis, atherosclerosis, cancer, cataracts, chronic obstructive pulmonary disease, diabetes, cellulitis, coronary artery disease, heart failure, hypertension, inflammatory bowel disease, macular degeneration, multiple sclerosis. Parkinson's disease, Reynaud's phenomenon, hepatitis C, reperfusion injury, MRSA, sepsis, folliculitis, gout, arthritis, hypoxia, hypoperfusion, hemorrhage, ethanol toxicity, shock, hepatic disease, diabetic ketoacidosis, exercise fatigue, non-Hodgkin's and Burkitt's lymphoma, nausea, vomiting, hyperventilation, abdominal pain, lethargy, shock, severe anemia, hypotension, irregular heart rhythm, tachycardia, weight gain, fibromyalgia, cardiovascular disease, respiratory disease, infection, diabetes, cellulitis and pancreatic impairment and infection.

Alkaline water, or water with a high concentration of OH− ions may be manufactured by any means generally used. In some embodiments, alkaline water may be manufactured by combining oxides and hydroxides including, but not limited to, calcium hydroxide, calcium oxide, sodium hydroxide, sodium oxide, potassium hydroxide, potassium oxide, magnesium hydroxide, or magnesium oxide, with water to create a non-corrosive base solution with a high concentration of OH− ions. The water may be tap, spring, mineral-free, filtered, purified, distilled, or any other suitable water with a low mineral concentration.

In some embodiments, the OH⁻ solution of the present invention may be formed through the dissolution of calcium hydroxide in water. In some embodiments, the calcium may be between 2 and 10% mole weight, preferably between 2 and 6% mole weight, more preferably 4% mole weight in water. Dissociation of the calcium hydroxide in water may be facilitated by any means applicable. In some embodiments, the calcium hydroxide solution may be agitated. In other embodiments, the calcium hydroxide solution may be exposed to a magnetic field. In further embodiments, the calcium hydroxide solution may be agitated while being exposed to a magnetic field.

Such manipulation of the solution will yield substantial dissolution of the calcium hydroxide, creating a supersaturated solution. In some embodiments, substantial dissolution is such that the dissociation of the calcium hydroxide is increased to between 50 and 95% of maximum dissociation, preferably between 50 and 75% of maximum dissociation, more preferably between 75 and 95% of maximum dissociation, in some cases greater than 95% dissociation. By maximum dissociation is meant that when additional calcium hydroxide is added to the solution at a given temperature or pressure, the calcium hydroxide precipitates out regardless of the length of time or additional agitation. In some embodiments, agitation of the calcium hydroxide solution in a magnetic field increases the pH of the calcium hydroxide solution to at least one pH unit higher than a normal saturated Ca(OH)₂ solution, in some embodiments even 1 to 3 pH units higher than a normal saturated Ca(OH)₂ solution. In further embodiments, agitating the solution in a strong magnetic field increases the solubility of the Ca(OH)₂ to greater than normal, preferably 2-200 times greater than normal, more preferably 50 to 100 times greater than normal, preferably 100 times greater than normal.

The magnetic field to which the calcium hydroxide solution is exposed may be generated by any means applicable. In some embodiments, the magnetic field may be generated by magnets, magnetic water treatment units or other magnetic field generating apparatus. Such magnetic field generating apparatus may be composed of one or a plurality of magnets which may surround, be placed around, or be otherwise disposed of adjacent to the container containing the Ca(OH)₂ solution. Any kind of magnet or apparatus that creates a strong magnetic field may be used. Magnets which may be used as part of magnetic water treatment units or to otherwise generate a magnetic field include, but are not limited to, NdFeB (Neodymium-Iron-Boron). Ferrite, AlNiCo (Aluminum-Nickel-Cobalt), SmCo (Samarium Cobalt), Alcomax (alloy of iron, nickel, aluminium, cobalt and copper), Cunife (copper, nickel and iron or copper, nickel, iron and cobalt), and Fernico (iron, nickel, cobalt) magnets. The magnets may be monopolar or bipolar. In other embodiments, the magnetic field generating apparatus may comprise electromagnets. In additional embodiments, the magnets may be encased in a housing. Such a housing may be made of any material applicable, including, but not limited to, metals such as, but not limited to, aluminum, or steel; and plastics, or any combination thereof. In some embodiments, magnets on opposing sides of the container holding the solution may have opposite poles, such that, for example, the positive and negative poles face each other. In other embodiments, the magnets may rotate around the container of calcium hydroxide solution.

In some embodiments, in order to increase the OH⁻ concentration of a calcium hydroxide solution, it may be combined with a solution made from sulfuric acid. In order to create the solution made from sulfuric acid, sulfuric acid is added to water. In some embodiments, enough sulfuric acid is added to water to create a solution of equal molar strength to the Ca(OH)₂ in the calcium hydroxide solution. In other embodiments, the concentration of the solution will be about 0.02% to about 0.08% acid in water by volume, preferably about 0.04% to about 0.06% acid in water by volume. In further embodiments the concentration may be about 50-100 ml of sulfuric acid (Baume 12°) per gallon of water, preferably about 70 to about 80 ml, more preferably about 70 to about 78 ml of sulfuric acid per gallon of water. In some embodiments, the sulfuric acid solution may be agitated until substantial dissociation occurs such that 75 to 100% of maximum dissociation is achieved, preferably 75 to 95% of maximum dissociation., more preferably 80 to 95% of maximum dissociation of sulfuric acid, in some instances greater than 95% dissociation of sulfuric acid.

In some embodiments, it may be desirable to reduce the acidity of the sulfuric acid solution. The reduction of acidity may occur through any means applicable. In some embodiments, the reduction of acidity may occur through the introduction of additional oxygen to the solution. In one embodiment, nascent oxygen may be introduced into the sulfuric acid solution. In another embodiment, the sulfuric acid solution may be treated with ozone by circulating the solution through ozone generators. The ozone generators dissociate an oxygen which is consumed by (2 H+) ion(s) in the acid solution to create water. The acid solution may be re-circulated through the ozone units until a particular concentration of oxygen is absorbed or a particular pH is achieved. In some embodiments, the sulfuric acid solution will be run through the ozone generators until the pH increases by at least 1 to 6 points, preferably at least 1 to 4 points, more preferably at least 2 to 3 points. In some embodiments, the sulfuric acid solution will be circulated through ozone generators until the pH reaches or exceeds about 7.0. The neutralized acid solution may then be slowly added to the calcium hydroxide solution to form a resultant mixture. The free calcium in the calcium hydroxide solution will react with the sulfate ions (SO₄²⁻) in the acid solution to create insoluble anhydrous calcium sulfate precipitate. The mixture may then be agitated until the reaction goes to completion and the anhydrous calcium sulfate may be filtered or otherwise removed from the solution. In some embodiments, a non-ionic surfactant may be added to the resulting mixture in order to enhance precipitation. Such non-ionic surfactants may include, but are not limited to. linear or nonyl-phenol alcohols or fatty acids, alcohol ethoxylates, alkylphenol ethoxylates, alkyl polyglycosides, alkyl ethers such as polyoxyethylene octyl ether, polyoxyethylene lauryl ether, polyoxyethylene stearyl ether, and polyoxyethylene oleyl ether; alkyl phenyl ethers such as polyoxyethylene octylphenyl ether, and polyoxyethylene nonylphenyl ether; alkyl esters such as polyoxyethylene laurate, polyoxyethylene stearate, and polyoxyethylene oleate; alkylamines such as polyoxyethylene laurylamino ether, polyoxyethylene stearylamino ether, polyoxyethylene oleylamino ether, polyoxyethylene soybean amino ether, and polyoxyethylene beef tallow amino ether; alkylamides such as polyoxyethylene lauric amide, polyoxyethylene stearic amide, and polyoxyethylene oleic amide; vegetable oil ethers such as polyoxyethylene castor oil ether, and polyoxyethylene rapeseed oil ether; alkanolamides such as lauric acid diethanolamide, stearic acid diethanolamide, and oleic acid diethanolamide; and sorbitan ester ethers such as polyoxyethylene sorbitan monolaurate, polyoxyethylene sorbitan monopalmitate, polyoxyethylene sorbitan monostearate, and polyoxyethylene sorbitan monooleate.

In other embodiments, the OH⁻ solution of the present invention may be formed through the dissolution of calcium oxide in water. In some embodiments, the calcium may be between 2 and 10% mole weight, preferably between 2 and 6% mole weight, more preferably 4% mole weight in water. Dissociation of the calcium oxide in water may be facilitated by any means applicable. In some embodiments, the calcium oxide solution may be agitated or stirred in order to increase the rate or amount or dissociation of the calcium oxide.

In some embodiments, the solution, however formed, may be filtered at various stages to remove particulates. For example, the calcium hydroxide solution may be filtered prior to combining with the sulfuric acid solution and/or the resultant mixture may be filtered to remove particulates. In other embodiments, the resultant mixture may be additionally cooled or partially frozen to create a slurry and further purified. For example through filtration. In one embodiment, the resulting mixture is cooled to below about 36° F. In another embodiment the resulting mixture is cooled to below about 36° F. but above about 35° F.

In some embodiments, the concentrated OH⁻ solution prepared by combining the calcium hydroxide solution and sulfuric acid solution or dissolution of calcium oxide may be diluted with water to reach a specified pH prior to consumption or administration. In some embodiments, the water may be non-chlorinated. In other embodiments, the water may be spring water. In further embodiments, the water may be distilled. In yet another embodiment, the water may be mineral free water. In additional embodiments, the water may be generated by an alkaline water machine. In some embodiments, the resulting mixture may be diluted to a pH of between about 8.0 to about 11, more preferably between about 8.5 to about 9.5, more preferably between 8.5 to about 9.0. This solution may then be used to effectively neutralize acids, to treat acidosis, prophylactically, to reduce free radicals, and/or as an antioxidant.

In some embodiments, calcium oxide (CaO), another strong base, is used as the acid neutralizing agent and/or antioxidant. Calcium oxide is stirred into purified, distilled, or mineral free water to create a non-corrosive base solution with a high concentration of OH⁻ ions. Such a solution will have a pH between about 7 to about 14, preferably a pH of about 12 to about 14, more preferably about 12.3 to about 13.8, preferably a pH of about 12.5 to about 13.75 and an ionic concentration such that the conductivity would be about 50 μS/cm to about 2000 μS/cm, preferably between about 100 μS/cm to about 1000 μS/cm, preferably about 500 μS/cm to about 800 μS/cm, preferably about 650 μS/cm to about 750 μS/cm, preferably about 700 μS/cm to about 750 μS/cm. In some embodiments, the ionic concentration is such that the conductivity is between 700 μS/cm to about 2000 μS/cm.

The resulting concentrated alkaline water may be consumed directly or diluted with water to a pH of about 7 to about 10, about 7.5 to about 8. The diluted alkaline water may have a conductivity of about 45μS/cm to about 90 μS/cm, about 50 μS/cm to about 75 μS/cm, about 50 μS/cm to about 60 μS/cm. In some embodiments, the water may be non-chlorinated. In other embodiments, the water may be spring water. In further embodiments, the water may be distilled. In yet another embodiment, the water may be mineral-free water. In additional embodiments, the water may be generated by an alkaline water machine.

In some embodiments, alkaline water may be taken in a concentrated formulation with a pH of between about 12 to about 13.75, preferably about 12 to about 12.5 and a conductivity of about 700 μS/cm to about 2000 μS/cm, preferably about 700 μS/cm to about 1500 μS/cm, preferably about 700 μS/cm to about 1000 μS/cm, more preferably about 700 μS/cm to about 750 μS/cm. In other embodiments, Alkaline water as described herein may be diluted with purified, distilled, tap, spring, non-chlorinated, or mineral free water to a pH of about 6.9 to about 7.5, preferably about 6.9 to about 7.2, more preferably about 7.0 and a conductivity 45 μS/cm to about 60 μS/cm, preferably about 50 μS/cm to about 55 μS/cm.

The acid/alkaline balance in a healthy mammal is generally regulated through the actions of buffers, respiration and renal function. Two forms of acid are generated as a result of normal metabolic processes. Oxidative metabolism produces a large amount of CO₂ daily which is excreted through the lungs. The other form of acid results from the metabolism of dietary protein, resulting in the accumulation at an average rate of approximately 1 mmol per kilogram of body weight, or 50 to 70 mmol per day of acid in an average adult on a typical western meat containing diet.

The most important mechanism preventing change in the pH of extracellular fluid is the carbonic acid/bicarbonate buffer system. The importance of this buffer pair relates to certain key properties: bicarbonate is present in a relatively high concentration in the extracellular fluid (between 24 and 28 mmol/L) and the components of the buffer system are effectively under physiological control: the CO₂ by the lungs, and the bicarbonate by the kidneys. A shift in pH can be brought about by either a primary change in the bicarbonate concentration (metabolic disturbances) or in the partial pressure of CO₂ in the blood (respiratory disturbances).

Respiratory acidosis results from the accumulation of CO₂ in the body as a result of failure of pulmonary ventilation. This may occur from lesions either in the central nervous system (e.g. depression of cerebral function, spinal cord injury), in the peripheral nervous pathways involved in ventilating the lungs (peripheral nerve and muscle disorders), in some forms of lung disease involving impaired gas diffusion (e.g. emphysema, asthma, bronchitis, pneumonia, lung cancer or aspiration), or due to pharmaceutical causes.

Metabolic acidosis may result from inorganic acid addition, i.e. the infusion or ingestion of HCl or NH₄Cl; or through gastrointestinal base loss through conditions such as diarrhea, small bowel fistula/drainage, surgical diversion, and renal tubular disorders; stimulation of chemoreceptors; lactic acid accumulation; poison; or diet. The OH⁻ solution of the present invention is effective in the treatment of acidosis regardless of cause.

Alkalinity increasing compositions of the invention typically comprise an amount of a base solution made from calcium hydroxide and/or calcium oxide, its solvates, hydrates, or combinations thereof, which is effective for the treatment or prevention of acidosis, as well as complications and related conditions thereof in a mammalian subject. Alkaline water may be taken alone, or in a coordinate or combined formulation with one or more additional agents to optimize health and performance; prevent illness; decrease recovery times from exertion, illness and injury; and extend endurance during exercise. Useful secondary or additional agents for use within the formulations and methods of the present invention include, but are not limited to, alkalinity increasing agents, adaptogens, amino acids and amino acid derivatives, anti-inflammatory agents, anti-nausea agents, analgesics, antioxidants, aphrodisiacs, detoxifying agents, dietary supplements, herbal supplements, calming agents, herbs and plant extracts, flavorings, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein. Within these formulations and methods, the secondary agent may be provided in any of a variety of forms, including any polymorphs, enantiomers, pharmaceutically acceptable salts, solvates, hydrates, or combinations thereof. Typically, an alkalinity increasing effective amount of an OH⁻ formulation will comprise an amount of the active compound which is therapeutically effective by itself or with one or more secondary agents, in a single or multiple unit dosage form, taken over a specified period of therapeutic intervention, to measurably alleviate one or more symptoms of acidosis or related conditions in the subject. Within exemplary embodiments, these compositions are effective within in vivo treatment methods to alleviate acidosis. The compositions described herein may additionally contain sweeteners, stabilizers, flavoring, anti-caking agents, flavor protectants, preservatives, anti-foaming agents, colorants, emulsifiers and the like.

Oxidative metabolism may also cause oxidative stress. Oxidative stress is imposed on cells as a result of an increase in oxidant generation (including reactive oxygen species), a decrease in antioxidant protection, or a failure to repair oxidative damage. It is believed that intracellular and extracellular advanced glycation (AGEs) or lipoxidation end products (ALEs), together with dysregulated glucose and lipid metabolism, are important contributors to oxidant stress, enhanced cellular redox-sensitive transcription factor activity, and impaired innate immune defense, causing inappropriate inflammatory responses mediated in part by reactive oxygen species.

Oxygen has two unpaired electrons in separate orbitals in its outer shell. Sequential reduction of molecular oxygen leads to the formation of a group of reactive oxygen species including the superoxide anion, peroxide and hydroxyl radicals. Oxygen-derived radicals are generated constantly as part of normal aerobic life as oxygen is reduced along the electron transport chain in mitochondria. Reactive oxygen species are also formed as necessary intermediates in a variety of enzyme reactions.

However, these highly reactive radicals can also start a chain reaction which disrupts cellular function. While they are a natural byproduct of metabolic function as well as part of phagocytosis, an excess of free radicals can occur for a variety of reasons. For example, an increase in the production of free radicals can be produced by drugs such as antibiotics that depend on quinoid groups or bound metals for activity (nitrofurantoin), antineoplastic agents as bleomycin, anthracyclines (adriamycin) and methotrexate. In addition, radicals derived from penicillamine, Phenylbutazone, some fenamic acids and the aminosalicylate component of sulphasalazine are currently believed to inactivate protease and deplete ascorbic acid accelerating lipid peroxidation. Free radical production may also be increased by radiation and smoking. Additionally, inhalation of inorganic particles also known as mineral dust (e.g. asbestos, quartz, and silica) can lead to lung injury due to free radical production. Fever, excess glucocorticoid therapy and hyperthyroidism also increase the generation of oxygen-derived radicals due to increased metabolism. Furthermore, a wide variety of environmental agents including photochemical air pollutants such as pesticides, solvents, anesthetics, exhaust fumes and aromatic hydrocarbons can cause free radical damage to cells.

Free radical and ROS damage can be inhibited by antioxidants. An antioxidant is a substance that when present in low concentrations relative to the oxidizable substrate significantly delays or reduces oxidation of the substrate. Antioxidants protect the body by reacting with free radicals and other reactive oxygen species within the body, hindering oxidation and reducing the amount of circulating free radicals. However, antioxidant supply is limited as an antioxidant molecule can only react with a single free radical. Therefore, there is a constant need to replenish antioxidant resources, whether endogenously or through supplementation. The compositions and methods of the present invention are effective as antioxidants for the elimination and/or reduction of reactive oxygen species including free radicals, regardless of the source of the free radicals.

Antioxidant compositions of the invention typically comprise an amount of a base solution made from calcium hydroxide or calcium oxide, its solvates, hydrates, or combinations thereof by itself or with one or more additional agents, which is effective for the treatment or prevention of excess free radicals as well as complications and related conditions thereof in a mammalian subject. Typically, an antioxidant effective amount (or free radical reducing effective amount) of an OH⁻ formulation of the present invention will comprise an amount of the active compound by itself or with one or more additional agents which is therapeutically effective, in a single or multiple unit dosage form, over a specified period of therapeutic intervention, to measurably alleviate one or more symptoms of free radical damage or related conditions in the subject. The compositions described herein may additionally contain sweeteners, stabilizers, flavoring, anti-caking agents, flavor protectants, preservatives, anti-foaming agents, colorants, emulsifiers and the like.

The amount, timing and mode of delivery of compositions of the invention comprising an effective amount of a base solution either as an alkalinity increasing agent, (antioxidant agent, free radical reducing agent) will be routinely adjusted on an individual basis, depending on such factors as weight, age, gender, and condition of the individual, the severity of the acidosis and/or free radical damage or related symptoms, whether the administration is prophylactic or therapeutic, and on the basis of other factors known to effect drug delivery, absorption, pharmacokinetics, including, but not limited to, half-life, and efficacy.

An effective dose or multi-dose treatment regimen for the instant alkalinity increasing or antioxidant formulations will ordinarily be selected to approximate a minimal dosing regimen that is necessary and sufficient to substantially prevent or alleviate acidosis or excess free radicals and related conditions in the subject. A dosage and administration protocol will often include repeated dosing therapy over a course of several days or even one or more weeks, months, or years. An effective treatment regime may also involve prophylactic dosage administered on a day or multi-dose per day basis lasting over the course of days, weeks, months or even years.

An “effective amount,” “therapeutic amount,” “therapeutic effective amount,” or “effective dose” is an amount or dose sufficient to elicit a desired pharmacological or therapeutic effect in a mammalian subject; typically resulting in a measurable increase in alkalinity or reduction in free radicals.

Therapeutic efficacy can alternatively be demonstrated by a measurement of blood gases, electron spin resonance, spin trapping, fingerprinting, measurement of free radical markers, liquid chromatography, measurement of markers of oxidative stress, lactic acid measurements, litmus tests, uric acid measurements, or by altering the nature, recurrence, or duration of conditions associated with acidosis and/or excess free radicals.

Therapeutic effectiveness may further be demonstrated by a decrease in the symptoms of the conditions being treated, for instance, a decrease in abscesses, boils, redness, pain, headache, a general sick feeling, muscle aches, shortness of breath. fatigue, fever, shivering and chest pain of mild to medium intensity, muscle aches, joint pain, bone pain, chest pain, painful breathing, shortness of breath, fever and chills, low blood pressure, fatigue, headaches, rash, malaise, septic shock, septic arthritis, abscesses deep within the body, blood poisoning, or septicemia, osteomyelitis, meningitis, endocarditis, pneumonia, joint inflammation, confusion, lethargy, rapid breathing, shortness of breath, wheezing, chest pain or pressure, joint stiffness, swelling, joint deformity, crepitus, non-specific fever, joint inflammation, headaches, fatigue, a feeling of euphoria, nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, memory loss, mood swings, musculoskeletal pain, easy bruising, nausea, vomiting, ascites, easy bruising, dry skin, dry mouth, low blood pressure, frequent urination, chest pain, lymph node pain, night sweats, skin rash, hyperventilation, constipation, severe anemia, and light headedness.

Therapeutic effectiveness may be determined, for example, through an arterial blood gas. In an arterial blood gas test, arterial blood is taken from any easily accessible artery (typically either radial, brachial or femoral) or out of an arterial line. Once the sample is obtained, care should be taken to eliminate visible gas bubbles, as these bubbles can dissolve into the sample and cause inaccurate results. The sealed syringe is then taken to a blood gas monitor. The machine aspirates the blood from the syringe and measures the pH and the partial pressures of oxygen and carbon dioxide and the bicarbonate concentration, as well as the oxygen saturation of hemoglobin. Normal pH of blood is between about 7.4 and 7.3, preferably 7.365. Effective amounts of the mixtures of the present invention will increase plasma from below 7.0 to a pH of about 7.6 to 7.3. Effective alkalinity increasing amounts may increase plasma pH of 6.0 to a pH of about 6.5, preferably to about 6.7, more preferably to about 7.0, preferably to a pH of 7.4 or higher.

Therapeutic effectiveness may also be demonstrated through a litmus test in which a sample of saliva is taken upon awakening and tested with a strip of litmus paper. A urine sample may also be tested with a strip of litmus paper or a litmus test strip. The litmus paper is then compared to a litmus scale to determine the pH of the sample. Optimally, the of saliva is about 7.4 and the pH of urine is about 6.6. The methods and compositions of the present invention are therapeutically effective to increase the pH of saliva and/or urine about 0.2 to about 3.2 units on the pH scale, about 0.4 to about 2 units on the pH scale, about 0.5 to about 1 unit or more on the pH scale.

Therapeutic effectiveness may also be determined using a Lactic acid meter. During intense exercise, physiological pH levels increase indicating an increase in acid in the body. The effect of alkaline water on lowering elevated physiological pH can be determined using a lactic acid meter. Measurements may be taken before, during, and after intense activity. An effective amount of an Alkaline water composition would maintain normal or decrease elevated levels of physiological pH during exercise. In some embodiments, alkaline water consumed during exercise will decrease the drop in physiological pH. In other embodiments, alkaline water consumed during exercise will prevent a drop in physiological pH. In additional embodiments, alkaline water consumed after exercise will increase the rate at which physiological pH returns to baseline levels. In some embodiments, the consumption of Alkaline water as described herein during exercise will increase an individual's maximal lactate steady state allowing them to exercise longer and harder than had previously been possible.

The normal blood lactate concentration is 1-0.5 mmol/L. Individuals in various disease states may have lactate concentrations of less than 2 mmol/L. Hyperlactemia is defined as a mild-to-moderate persistent increasing blood lactate concentration (2-5 mmol/L) without metabolic acidosis, whereas lactic acidosis is characterized by persistently increased blood lactate levels (usually >4-5 mmol/L) in association with metabolic acidosis. The Lactic threshold at which point exercise becomes more difficult is between 2 to 4 mmol/L. Consumption of an effective amount of an Alkaline water composition as described herein will delay an increase in lactic acid levels during exercise and/or increase the rate at which lactic acid levels return to normal levels after exercise. Consumption of an effective amount of Alkaline water as described herein will delay an increase in lactic acid levels during exercise by 5%, 10%, 20%, 30%, 50% or greater reduction, up to a 75-90%, or 95% or greater. Furthermore, consumption of an effective amount of Alkaline water as described herein will decrease elevated lactic acid levels by 5%, 10%, 20%, 30%, 50% or greater reduction, up to a 75-90%, or 95% or greater, reduction regardless of the cause of the elevation, whether exercise or illness.

Therapeutic effectiveness may also be determined with a uric acid meter. Normal uric acid is between 3.5 mg/dL to 7.2 mg/dL with 20 mg/dL being a major gout attack. Elevated levels of uric acid may be due to acidosis. Consumption of an effective amount of Alkaline water as described herein will decrease uric acid levels by 5%, 10%, 20%, 30%, 50% or greater reduction, up to a 75-90%, or 95% or greater.

The rate of Cytochrome C reduction can be measured using luminol induced chemiluminescence for quantifying the results. Therapeutically effective free radical reducing or antioxidant amounts of the solution of the present invention will decrease the rate of cytochrome C reduction by 2-50%, 10-40%, 15-30%, 20-25% or more, up to a 75-90%, or 95% or greater, reduction.

Following administration of the OH⁻ composition according to the formulations and methods of the invention, test subjects will exhibit a 5%, 10%, 20%, 30%, 50% or greater reduction, up to a 75-90%, or 95% or greater, reduction, in one or more symptoms associated with acidosis as compared to placebo-treated or other suitable control subjects. Test subjects may also exhibit a :10%, 20%, 30%, 50% or greater reduction, up to a 75-90%, or 95% or greater, reduction, in the symptoms of one or more conditions associated with acidosis.

The compositions of the present invention may be administered by any means that achieves the intended therapeutic or prophylactic purpose. Suitable routes of administration for alkalizing and antioxidant compositions of the invention comprising OH⁻ solutions include, but are not limited to, oral, buccal, nasal, aerosol, mucosal, injectable, slow release, controlled release, iontophoresis, sonophoresis, and other conventional delivery routes, devices and methods.

Within additional aspects of the invention, combinatorial formulations and coordinate administration methods are provided which employ an effective amount of OH⁻ compositions, and one or more additional active agent(s) that is/are combinatorially formulated or coordinately administered with the OH⁻ solution—yielding an effective formulation or method to modulate, alleviate, treat or prevent acidosis or excessive free radicals in a mammalian subject. Exemplary combinatorial formulations and coordinate treatment methods in this context employ a base solution in combination with one or more additional or adjunctive agent.

Such secondary or additional agents for use within the formulations and methods of the present invention include, but are not limited to, alkalinity increasing agents, adaptogens, amino acids and amino acid derivatives, anti-inflammatory agents, anti-nausea agents, analgesics, antioxidants, aphrodisiacs, detoxifying agents, dietary supplements, herbal supplements, calming agents., herbs and plant extracts, flavorings, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein. Within these formulations and methods, the secondary agent may be provided in any of a variety of forms, including any polymorphs, enantiomers, pharmaceutically acceptable salts, solvates, hydrates, or combinations thereof. Such combinations of an OH⁻ composition and secondary agent may be administered either combinatorially or coordinately as disclosed herein to effectively optimize health and performance; prevent illness; decrease recovery times from exertion, illness, and injury; increase energy levels; improve exercise performance; improve hydration; prevent muscle damage after exercise; and increase stamina during exercise.

Alkalinity increasing agents for use within the formulations and methods of the present invention include, but are not limited to sodium bicarbonate; a carbonate, a phosphate, or a hydroxide of sodium or potassium; magnesium carbonate; magnesium hydroxide; ammonium carbonate; ammonium bicarbonate; magnesium oxide; sodium or potassium citrate, bicarbonate, sulfate, and benzoate; ascorbate; calcium carbonate; any pharmaceutically acceptable material that causes the pH of an aqueous medium to rise above pH 7.0, or mixtures thereof.

Adaptogen agents for use within the formulations and methods herein include, but are not limited to, ashwagandha, eleutherococcus senticosus, reishi, astragalus, licorice root, panax quinquefolius, panax ginseng and schisandra berries.

Antioxidants included in the formulations provided herein may be in the form of nutritional supplements such as, but not limited to, vitamin A; vitamin C; vitamin E; erythorbic acid; beta-carotene; carotenes; lutein; manganese; lycopene; melatonin; or coenzyme Q10; xanthine oxidase inhibitors, including, but not limited to, allopurinol and folic acid; NADPH oxidase inhibitors, including, but not limited to, adenosine; calcium channel blockers; superoxide dismutases; catalases; albumin; inhibitors of iron redox cycling, including, but not limited to deferoxamine, apotransferin and ceruloplasmin; beta carotene; ascorbates; myricetin-3-O-galactoside, quercitin-3-O-galactoside; alpha tocopherol; and benzaldehyde derivatives, such as those described in U.S. patent application Ser. No. 12/418,342, incorporated by reference herein in its entirety. In some embodiments, antioxidants may be present in plant extracts which may also be combined with the alkaline water. Plant extracts may come from plant sources such as, but not limited to, apricot, acai fruit, acerola, apple, blueberry, blackberry, black currant, carrots, cherry, chokeberry, cranberry, elderberry, green tea, goji berry, grape seed, mangosteen, maqui berry, milk thistle, pomegranate seed, prune, raspberry, red grape, rooibos, rosehips, strawberry, seabuckthorn, white grape, whole grape, yumberry and acerola fruit.

Vitamin, mineral and nutritional supplements for use herein may be in a variety of forms including, but not limited to, vitamin B complex, folic acid, niacin, niacinamide, pantothenic acid, pyridoxine HCl, vitamin B2, folate, biotin, vitamin C, vitamin D, vitamin D₃, vitamin E, vitamin K, cyanocobalamin, inositol, thiamine, thiamine mononitrate, calcium pantothenate, mixed tocophyerols, d-alpha tocopheryl acetate, magnesium, calcium, calcium carbonate, calcium chelate, calcium di-phosphate, calcium phosphate, iron, magnesium carbonate, magnesium citrate, magnesium oxide, magnesium phosphate, manganese chelate, manganese sulfate, potassium, potassium chelate, potassium chloride, sodium, zinc, vanadyl sulphate, chromium, chromium chloride, chromium picolinate, and chromium polynicotinate.

Amino acids, amino acid precursors and derivatives as used within the formulations herein may be branched or straight chain amino acids. Exemplary amino acids, precursors and derivatives which may be used in the formulations and methods described herein include, but are not limited to, 5-HTP, arginine, beta alanine, carnitine fumarate, citrulline malate, glutamine peptide, glycine, 1-alanine, 1-arginine, 1-arginine hydrochloride, 1-histidine, 1-methionine, 1-lysine Mt, 1-phenylalanine, leucine ethyl ester, 1-glutamine, 1-isoleucine, 1-theanine, 1-tyrosine, phenylalanine, taurine, tri-methyl glycine, tryptophan, tyrosine, 1-carnitine, 1-carnosine, glutamine alpha ketoglutarate and alpha-L-poly lactate.

Electrolytes used with the formulations herein include, but are not limited to, sodium chloride, sodium acetate, acidic sodium citrate, acidic sodium phosphate, sodium chloride, sodium bicarbonate, sodium bromide, sodium citrate, sodium lactate, sodium molybdate, sodium phosphate, anhydrous sodium sulphate, sodium sulphate, sodium tartrate, sodium benzoate, sodium selenite, and other sodium salts and mixtures thereof; potassium chloride, potassium acetate, potassium bicarbonate, potassium bromide, potassium citrate, potassium-D-gluconate, monobasic potassium phosphate, potassium tartrate, potassium sorbate, potassium iodide, and other potassium salts and mixtures thereof; magnesium carbonate, magnesium citrate, magnesium oxide, magnesium phosphate, as well as other magnesium salts and mixtures thereof; calcium chloride, calcium carbonate, calcium chelate, calcium di-phosphate, calcium lactate, calcium phosphate tribasic and other calcium salts and mixtures thereof. Such electrolytes may be included in the formulations described herein in proportions and amounts suitable to replenish salts lost during exercise or illness or otherwise depleted.

Anti-inflammatory agents for use within the formulations and methods herein include, but are not limited to, extracts from plants such as maqui berry, milk thistle, skull cap, red raspberry, red sour cherry, green tea and hops.

Other agents which may be used in the compositions and methods described herein include anti-nausea agents including, but not limited to, extracts from peppermint, ginger and chamomile.

A further agent which may be used in the compositions and methods described herein includes analgesic agents such as, but not limited to, white willow hark.

The formulations and methods described herein may additionally include herbal supplements and extracts with beneficial properties including, but not limited to, passion flower, horny goat weed, skullcap, milk thistle, Echinacea, dandelion leaf St. John's wort, green tea, black tea, chamomile or peppermint, or an extract thereof.

The formulations and methods described herein may further include plants with beneficial properties including, but not limited to, guarana seeds, acerola berries, coconut water, verba mate, acai berry, ginseng root, panax ginseng root, ginkgo biloba, white willow bark, acacia, ashwagandha, chokeberry, elderberry, cranberry, maqui berry, blueberry, pomegranate, rooibos, goji berry, elder berry, valerian, seabuckthorn, yumberry, blackberry, astragalus, damiana, and ginger.

Energy boosters that may increase performance and are contemplated for use within the methods and formulations described herein include, but are not limited to, creatine ethyl ester, creatine monohydrate, magnesium creatine chelate, creatine hydrochloride, creatine nitrate, creatine monohydrate and royal jelly.

Useful flavonoids within the compositions and methods of the present invention are present in chamomile extract, cocoa powder, red grape, black tea, and white tea, ginkgo biloba, berries, parsley, and green tea some or all of which may be included in the compositions and methods described herein.

Useful sedatives for use within the compositions and methods described herein include, but are not limited to, lavender, lemon balm, lemongrass, linden. oatstraw, St. John's wart, valerian root, kava kava, hops and passion flower.

Stimulants for use within the methods and compositions described herein include, but are not limited to, caffeine, citicoline, d-glucuronolactone, guarana extract, ginseng, concentrated green tea, green coffee beans, glucuronolactone, guarana, panax ginseng, panax quinquefolius, Siberian ginseng, and theobromine.

Additional agents which may be included in the formulations and methods described herein are immune boosters including, but not limited to, Echinacea and astragalus root.

Flavoring agents for use with the compositions and methods described herein include, but are not limited to fruit juice, vegetable juice, milk solids, fruit flavors, herbal flavor and mixtures thereof. The fruit juice can be any citrus juice, non-citrus juice, or mixture thereof, which is known for use in dilute juice beverages. The juice can be derived from, but not limited to, apple, cranberry, pear, peach, plum, apricot, nectarine, grape, guava, cherry, currant, raspberry, gooseberry, elderberry, blackberry, blueberry, strawberry, lemon, lime, mandarin, orange, tomato, lettuce, dandelion, rhubarb, pineapple, coconut, pomegranate, kiwi, mango, papaya, banana, watermelon, passion fruit, tangerine, and cantaloupe. The vegetable juice can be any vegetable juice generally consumed including but not limited to, celery, spinach, cabbage, watercress, carrot, beet, spirulina, sweet potato, kale, romaine, collard greens, endive, escarole, bok choy, fennel, parsley, wheat grass, or cucumber. Such fruit and vegetable juices may or may not have additional beneficial properties such as antioxidants and/or flavonoids.

Formulations and methods herein may additionally include a protein source. Protein sources include, but are not limited to, milk solids, calcium caseinate, whey protein concentrate, whey protein isolate, whey protein hydrolysate, soy protein, casein hydrolysate, rice protein, wheat protein, corn protein, partially hydrolyzed whey protein, or ultra-filtered whey protein.

Formulations and methods herein may further include one or more sweeteners or other carbohydrate source. Such sweeteners include, but are not limited to, acesulfame potassium, aspartame, cane sugar, corn syrup, crystalline fructose, dextrose, D-ribose, fructose, glucose, glucose-fructose syrup, high fructose corn syrup, high fructose liquid sugar, honey, maltodextrin, sorbitol, stevia, sucralose. sucrose, sugar, trehalose, truvia or xylitol.

Further additional or adjunctive therapeutic agents may include but are not limited to, probenecid, allopurinol, nonsteroidal anti-inflammatory drugs (NSAIDs), colchicine, corticosteroids, uricosuric agents, xanthine oxidase inhibitors, losartan, lenofibrate, urate oxidase, Y-700, COX-2 inhibitors, analgesics, corticosteroids. disease-modifying anti-rheumatic drugs, antibiotics, vasodepressors, sulfasalazine, radiation therapy, chemotherapy, duloxetin, milnacipran, gabapentin, pregabalin, and benzaldehyde derivatives such as those described in U.S. patent application Ser. No. 12/418,342, incorporated herein by reference in its entirety.

The use of these additional or adjunctive agents in conjunction with the alkalizing or antioxidant agent of the present invention may increase the effectiveness of the therapeutic agents and/or decrease the amount of such agents that may be required to optimize health.

In some embodiments, the alkalinity increasing agent may be administered in conjunction with an additional therapeutic agent to facilitate consumption of the additional therapeutic agent. For example, some therapeutic agents may be extremely acidic. Such agents may be administered in conjunction with the alkalinity increasing agent to neutralize the acidity and increase the forms of administration that would be acceptable. In another embodiment, the alkalinity increasing agent may be used to temporarily neutralize stomach acid or other acid conditions so that therapeutic agents which are destroyed by acid such as, but not limited to, nutritional supplements or other organics such as vitamins, including vitamin B₁₂, can be ingested.

In certain embodiments, the invention provides combinatorial alkalizing or antioxidant formulations comprising a base solution made from calcium hydroxide and/or calcium oxide and one or more adjunctive agent(s) having alkalizing or antioxidant activity, or both, or additional adjunctive agents which may have neither alkalizing nor antioxidant activity but which are useful in the treatment of underlying conditions or prophylactically. Within such combinatorial formulations, the OH⁻ solution and the adjunctive agent(s) having alkalizing and/or antioxidant activity, or non-alkalizing/antioxidant agents will be present in a combined formulation in effective amounts, alone or in combination. In exemplary embodiments, a base solution and a non-calcium hydroxide based alkalizing and/or antioxidant agent will each be present in an alkalizing and/or antioxidant amount (i.e., in singular dosage which will alone elicit a detectable alkalizing or free radical reduced response in the subject). Alternatively, the combinatorial formulation may comprise one or both of the OH⁻ solution and a non-calcium hydroxide based alkalizing and/or antioxidant agent or other adjunctive agent in sub-therapeutic singular dosage amount(s), wherein the combinatorial formulation comprising both agents features a combined dosage of both agents that is collectively effective. Effectiveness may elicit an alkalizing or free radical reducing response or other increased therapeutic response. Thus, one or both of the OH⁻ solution and a non-calcium hydroxide based alkalizing and/or antioxidant agents may be present in the formulation, or administered in a coordinate administration protocol, at a sub-therapeutic dose, but collectively in the formulation or method they elicit a detectable alkalizing and/or antioxidant response in the subject.

To practice the coordinate administration methods of the invention, an OH⁻ mixture is administered, simultaneously or sequentially, in a coordinate treatment protocol with one or more of the secondary or adjunctive therapeutic agents contemplated herein. The coordinate administration may be done simultaneously or sequentially in either order, and there may be a time period while only one or both (or all) active agents, individually and/or collectively, exert their biological activities. A distinguishing aspect of all such coordinate treatment methods is that the OH⁻ solution exerts at least some detectable alkalizing or antioxidant activity, and/or elicits a favorable clinical response, which may or may not be in conjunction with a secondary clinical response provided by the secondary therapeutic agent. Often the coordinate administration of a base solution with a secondary agent as contemplated herein will yield an enhanced therapeutic response beyond the therapeutic response elicited by either or both the OH⁻ solution and/or secondary therapeutic agent alone.

The amount, timing and mode of delivery of compositions of the invention comprising an effective amount of a base solution of the present invention will be routinely adjusted on an individual basis, depending on such factors as weight, age., gender, and condition of the individual, the severity of the acidosis, ROS levels including free radical production or related symptoms, whether the administration is prophylactic or therapeutic, and on the basis of other factors known to effect drug delivery, absorption, pharmacokinetics, including, but not Limited to, half-life, and efficacy.

Effective doses may be extrapolated from dose-response curves derived from in vitro or animal model test systems. Such animal models and systems are well known in the art. The precise dose to be employed will also depend on the route of administration, the seriousness of the disease or disorder, and body size, and should be decided according to the judgment of the practitioner and each patient's circumstances. However, suitable dosage ranges for oral administration are generally about 5 ounces (0.147 L) to about 135.256 ounces (4 L) of the diluted OH⁻ solution (having a pH between 7.5 and 9.5) per day. In specific preferred embodiments of the invention, the oral dose is about 5 ounces (0.147 mL) to about 100 ounces (2.9 L), about 5 ounces (0.147 L) to about 90 ounces (2.6 L) of OH⁻ solution per day, more preferably about 8 ounces (236 mL) to about 80 ounces (2.36 L) of OH⁻ solution per day, more preferably about 24 (0.7 L) to about 32 ounces (0.94 L) per day, more preferably about 32 ounces (0.94 L) to about 48 ounces (1.4 L) per day, more preferably about 35 ounces (1.035 L) to about 80 (2.36 L) ounces per day. In some embodiments, the OH⁻ solution is administered over the course of a day, for example the dosage is taken over eight hours, ten hours, twelve hours or 24 hours. In some embodiments, the dose may be calibrated based on body size, with effective doses comprising between about 0.01 to about 5 oz/pound, 0.3 to about 5 oz/pound, about 0.3 to about 3 oz/pound, about 0.3 to about 1 oz/pound, about 0.35 oz/pound. For example, an individual weighing 225 lbs would be given a starting dose of about 80 ounces (2.36 L) of diluted OH− solution, as described in Example X; an individual weighing 180 pounds would receive a starting dose of 64 oz (1.9 L) of the solution of Example X per day. An individual weighing 135 pounds would receive a starting dose of 48 oz (1.4 L) of the solution of Example X per day. An individual weighing 90 pounds would be given a starting dose of 32 oz (0.94 L) of the solution of Example X per day. An individual weighing 45 pounds would be given a starting dose of 16 oz (0.47 L) of the solution of Example X per day and an individual weighing 22 lbs would be given a starting dose of 8 oz (0.236 L) of the solution of Example X per day. In other embodiments, fractions of the dosage are administered at particular time points, for example every hour, every two hours, every three hours, every four hours, every eight hours, every twelve hours, or any other fraction of time, as tolerated by the patient. In one embodiment, one ounce of anti-oxidant material could be mixed in 1 liter of water. In another embodiment, three ounces of anti-oxidant material would be mixed in two liters of water. In a further embodiment, once the desired physiological pH level is obtained, a maintenance dose may be taken indefinitely. In some embodiments, a maintenance dose may be ½ of the therapeutic level, ⅓ of the therapeutic level, ¼ of the therapeutic level, or any other reduced dosage as determined by the judgment of the practitioner and the patient's circumstances.

The formulations may be presented in unit-dose or multi-dose containers. Preferred unit dosage formulations are those containing a daily dose or unit, daily sub-dose, as described herein above, or an appropriate fraction thereof, of the active ingredient(s). In one embodiment eight ounces of the prepared formulation is administered every four hours. In another embodiment, eight ounces of the prepared formulation is administered every three hours. In a further embodiment, 0.5 L is administered every eight hours. In still another embodiment, eight ounces of the prepared formulation is administered every two hours or fraction thereof. In exemplary embodiments, unit dose formulations are in 0.5 liters, or a multiple thereof.

Dosage forms of the OH⁻ solution of the present invention include excipients recognized in the art of compounding as being suitable for the preparation of dosage units as discussed above. Such excipients include, without intended limitation, binders, fillers, lubricants, emulsifiers, suspending agents, sweeteners, flavorings, preservatives, buffers, and other conventional excipients and additives.

The compositions of the invention for altering physiological pH can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

Additional OH⁻ solutions of the invention can be prepared and administered in any of a variety of inhalation or nasal delivery forms known in the art. Devices capable of depositing aerosolized OH⁻ formulations in the sinus cavity or pulmonary alveoli of a patient include metered dose inhalers, nebulizers, sprayers, and the like. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of calcium hydroxide and any additional active or inactive ingredient(s).

Yet additional OH⁻ formulations are provided for parenteral administration, including aqueous and non-aqueous sterile injection solutions which may optionally contain antioxidants, buffers, bacteriostats and/or solutes which render the formulation isotonic with the blood of the mammalian subject; and aqueous and non-aqueous sterile suspensions which may include suspending agents and/or thickening agents.

The products within the scope of this invention may take a variety of forms. For example, the product may be manufactured and sold as a ready-to-drink beverage for immediate consumption by a mammalian subject. The compositions described herein may be preferred in concentrat4e or powder form to be reconstituted for use by a mammalian subject by the addition of water. Such reconstitution is made with the requisite amounts of water to ensure that the beverage to be consumed contains the active components in the proportions previously noted. In some embodiments, liquid formulations as described herein may be sold as part of a kit including a lactic acid meter, uric acid meter and/or pH test strips. The pH test strip would be effective between a pH of 4.5 and 9.0, with measurements in increments of 0.25. The kits of the present invention comprise one or more compositions of the present invention together with the lactic acid meter, uric acid meter and/or pH test strips, information which informs a user of the kit, by words, pictures, and/or the like, that use of the kit will provide one or more general health and/or general physiological benefits including, but not limited to, alkaline increasing, health and performance optimizing, illness preventing, energy level increasing, hydration increasing, recovery time decreasing, muscle protecting and stamina increasing benefits and which informs the user of the method of monitoring their acidosis levels. By way of example only, the kit may comprise 7 bottles of 0.5 L of diluted alkaline water at a pH of 7.5. In other embodiments, the kit may comprise 7 bottles of concentrated alkaline water in 30mL bottles at a pH of 12.5.

The invention disclosed herein will also be understood to encompass methods and compositions comprising a base solution using in viva metabolic products of the said compounds (either generated in viva after administration of the subject precursor compound, or directly administered in the form of the metabolic product itself). Such products may result, for example, from the oxidation, reduction, hydrolysis, amidation, esterification and the like of the administered compound, primarily due to enzymatic processes. Accordingly, the invention includes methods and compositions of the invention employing compounds produced by a process comprising contacting a base solution of the present invention with a mammalian subject for a period of time sufficient to yield a metabolic product thereof.

The above disclosure generally describes the present invention. A more complete understanding can be obtained by referring to the following examples. These examples are described solely for purposes of illustration and are not intended to limit the scope of the invention. Although specific terms have been employed herein, such terms are intended for descriptive use and not for purposes of limitation.

### EXAMPLES

As demonstrated in the examples below, the present invention relates to the creation of a strong base solution for use as an antioxidant and/or alkalinity increasing agent.

### Example I

### Preparation of Basic Solution

50,000 g of Ca(OH)₂ is added to 500 gallons of water (100 g/gal) in a polyurethane tank surrounded by strong mono-polar magnets. The mixture is stirred until maximum disassociation is achieved. The solution is then passed through a 10 micron filter to remove any particulates. 78 ml of concentrated sulfuric acid (Baume 12°) per gallon, (39000 ml total) is added to a second polyurethane tank containing 500 gallons of pure water. The acid solution is circulated through an OzoTech OZ2PCS ozone generator (OzoTech, Inc., Yreka, Calif.) until the pH of the solution is above 7.0. The diluted sulfuric acid is then added to the filtered Ca(OH)₂ solution and the reaction is allowed to go to completion. The resulting solution is passed through a 10 micron filter to remove any anhydrous calcium sulfate.

### Example II

### Additional Purification of Ca(OH)₂ Solution

The solution of example I is chilled to below 36° F. for up to four hours, but not allowed to freeze completely. The partially frozen material is then filtered using a 6 micron filter to remove any newly precipitated anhydrous calcium sulfate and/or ice. This increases the negative charge and the molar strength of the solution.

### Example III

### Preparation of an Antioxidant Solution

The solution of example I is added to non-chlorinated drinking water until a pH of 8.5 to 9.0 is achieved.

### Example IV

### Treatment for Increasing Physiological pH

The solution of Example III was administered at the rate of 8 ounces every four hours until 24 to 32 ounces of the solution was consumed. Consumption of this amount increases physiological pH to normal levels and decreases rates of infection.

### Example V

### Preparation and Use of an Antioxidant Solution

An alcohol extraction of Dwarf Mistletoe, Arceuthobium campyopodum, is prepared to extract myricetin-3-0-galactoside and quercitin-3-0-galactoside. The berries of the Dwarf Mistletoe are harvested and then ground into a coarse powder. The powder is then placed in an Erlenmeyer flask with 80% cold methanol. After 24 Hours, the methanol is decanted and saved, and a second aqueous extraction is carried out for a further 24 hours. The combined methanol eluents are evaporated under vacuum leaving an aqueous solution. A half ounce of the aqueous solution is then combined with 1 liter of the solution of Example I which has been diluted to a pH of 11. The resulting solution may then be taken over 8 hours.

### Example VI

### Preparation of Alkaline Water Solution Using Calcium Oxide

3.2 g of calcium oxide (CaO) was added to one liter of distilled or mineral free water. The mixture was stirred for approximately ten minutes and filtered with a non-charcoal five micron filter resulting in water with a total alkalinity of 2000 mg/L CaCO₃.

### Example VII

### Preparation of Extra Strength Alkaline Water Solution Using Calcium Oxide

4 grams of calcium oxide (CaO) were added to one liter of distilled or mineral free water. The mixture was stirred for approximately ten minutes and then filtered with a non-charcoal five micron filter resulting in water with a 2400 mg/L CaCO₃ total alkalinity.

### Example VIII

### Preparation and Use of Concentrated Calcium Oxide Based Alkaline Water Solution

1 oz of the concentrate of Example VI or VII was diluted in 32 oz of purified, demineralized or distilled water. A mammalian subject consumes 8 ounces every four hours until 24 to 32 ounces of the solution is consumed. Consumption of this amount increases physiological pH to normal levels and decreases rates of infection.

### Example IX

### Preparation of Concentrated Alkaline Water Using Calcium Oxide-Version 2

Five gallons of filtered water are added to a non-reactive drum fitted with a bucket mixer. While stirring, calcium oxide is added until the solution reaches a pH of 12.75 pH as determined by a Waterproof EcoTestr pH 2 (Oakton Instruments, Vernon Hills, Ill.) and has a conductivity of between 700 to 750 μS as determined by COM-100: Waterproof EC/TDS/Temp Combo Meter (HM Digital, Inc., Culver City, Calif.) The resulting solution is then filtered with a non-charcoal live micron filter and decanted into 30 mL, containers for distribution.

### Example X

### Dilution of Alkaline Water for Consumption

Five gallons of filtered water are added to a non-reactive drum fitted with a bucket mixer. While stirring, calcium oxide is added until the solution reaches 12.75 pH as determined by a Waterproof EcoTestr pH 2 (Oakton Instruments, Vernon Hills, Ill.) and has conductivity between 700 to 750 μS as determined by COM-100: Waterproof EC/TDS/Temp Combo Meter (HM Digital, Inc., Culver City, Calif.). 30 mL of the solution is put in a second non-reactive drum fitted with a bucket mixer and water added. The resulting solution has a pH of 7 to 7.5 or slightly higher and has a conductivity of 50 μS as determined by COM-100: Waterproof EC/TDS/Temp Combo Meter (HM Digital, Inc., Culver City, Calif. The solution is filtered with a non-charcoal five micron filter and then decanted into 16.9 oz (0.5 L) containers for consumption.

### Example XI

### Preparation of Alkaline Water Using Sodium Hydroxide

Five gallons of filtered water are added to a non-reactive drum fitted with a bucket mixer. While stirring, sodium hydroxide is added until the solution reaches a pH of 12.5 pH as determined by a Waterproof EcoTestr pH 2 (Oakton Instruments, Vernon Hills, Ill.) and has a conductivity of between 700 to 750 μS as determined by COM-100: Waterproof EC/TDS/Temp Combo Meter (HM Digital, Inc., Culver City, Calif.).

### Example XII

### Preparation of Alkaline Water Using Calcium Oxide—Version 3

In a 5 gallon non-reactive drum fitted with a mixing device, 9.5 g of calcium oxide is added to 18.9 liters of distilled water and thoroughly mixed at 26.6° C. The pH of the resulting mixture is measured using a Waterproof EcoTestr pH 2 (Oakton Instruments, Vernon Hills, Ill.). If the pH is less than 12.5, calcium oxide is added by milligrams until the desired pH is achieved. The conductivity of the resulting solution is then measured using a COM-100: Waterproof EC/LDS/Temp Combo Meter (HM Digital, Inc., Culver City, Calif.) and is between 700 and 750 μS.

### Example XIII

### Preparation of Concentrated Alkaline Water Using Calcium Oxide—Version 4

In a 1.5 glass liter beaker, 500 mg of calcium oxide is added to 1 liter of water and mixed thoroughly at 26.6° C. The pH of the resulting mixture is measured using a Waterproof EcoTestr pH 2 (Oakton Instruments, Vernon Hills, Ill.). If the pH is less than 12.5, calcium oxide is added by milligrams until the desired pH is achieved. The conductivity of the resulting solution is then measured using a COM-100: Waterproof EC/TDS/Temp Combo Meter (HM Digital, Inc., Culver City, Calif.) and is between 700 and 750 μS.

### Example XIV

### Determination of Effectiveness of Alkaline Water

Ten (10) healthy male subjects ranging from 25 to 35 years old are given 2 L of water in 0.5 L doses as a placebo for three days. They are then given 2 L of Alkaline water in 0.5 L doses as prepared in Example X daily for two weeks. On days three, seventeen, and eighteen subjects undergo aerobic performance assessment on a stationary exercise bicycle in which power is increased from 25 watts to 175 watts at 25 watt intervals. Each interval lasts three minutes. At the end of each peripheral blood is collected to measure lactic acid and anaerobic tolerance. Peripheral blood is collected at 5 minutes, 30 minutes, 1 hour and 24 hours after the last interval. Subjects have continuous heart rate VO₂ and CO₂ monitoring. Lactic acid levels are measured using a Lactic Acid meter (Sports Resource Group (Hawthorne, N.Y.)).

### Example XV

### Determination of Change in Lactic Acid Threshold

Ten (10) healthy males participate in this trial. Subjects warm up for 1:5 minutes on a stationary bike and then work to their peak sustained intensity within the first 10 minutes and continue for twenty minutes. Using a heart rate monitor, the average heart rate is calculated over the last 20 minutes. Each subject is then given 2 L of Alkaline water in 0.5 L doses taken four times a day as prepared in Example X daily for two weeks. After two weeks, the subjects are retested and the average heart rate (estimated heart rate at subject's lactate threshold) is compared.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Garrett, William E. and Donald T. Kirkendall, Exercise and Sport Lippincott Williams & Wilkins; 1st edition (Jan. 15, 2000) p. 61
McArdle, W. D., Katch, F. I., & Katch, V. L. 1996. Exercise Physiology: Energy, Nutrition, and Human Performance. Baltimore, Md.: Williams & Wilkins.
Robergs, R. A., Ghiasvand, F., Parker, D. (2004). Biochemistry of exercise-induced metabolic acidosis. American Journal of Physiology: Regulatory, Integrative and Comparative Physiology. 287: R502-R516.
Robergs, R. A., & Roberts, S. 1997. Exercise Physiology: Exercise, performance, and clinical applications. St Louis, Mo.: Mosby.

We claim:

1. A method for preparing a resultant mixture having a high concentration of OH− ions comprising:
a) preparing a first solution by adding calcium oxide to water;
b) agitating the first solution to increase a rate or amount of dissociation of the calcium oxide;
c)measuring the pH of the first solution; wherein if the pH of the solution is less than 12.5, an additional amount of calcium oxide is added to the first solution to create a second solution;
d) agitating the second solution to increase a rate or amount of dissociation of the calcium oxide; and
e) measuring the pH of the second solution to determine that it has a pH of about 12.5.

2. The method of claim 1, wherein the second solution has a conductivity from about 700 μS/cm to about 2000 μS/cm.

3. The method of claim of claim 1, wherein the second solution has a conductivity from about 700 μS/cm to about 750 μS/cm.

4. A method for preparing a resultant mixture having a high concentration of OH− ions Comprising:
a) preparing a first solution by adding calcium oxide to water;
b) agitating the first solution to increase a rate or amount of dissociation of the calcium oxide;
c)measuring the pH of the first solution; wherein if the pH of the solution is less than 12.5, an additional amount of calcium oxide is added to the first solution to create a second solution;
d) agitating the second solution to increase a rate or amount of dissociation of the calcium oxide;
e) measuring the pH of the second solution to determine that it has a pH of about 12.5; and
f) diluting the second solution with water so that it has a pH of about 7.0-7.5.

5. The method of claim 4, wherein the resultant mixture has a conductivity of about 60 μS/cm.

6. A method of increasing alkalinity in a mammalian subject suffering from acidosis comprising:
a) preparing a first solution by adding calcium oxide to water;
b) agitating the first solution to increase a rate or amount of dissociation of the calcium oxide;
c)measuring the pH of the first solution; wherein if the pH of the solution is less than 12.5, an additional amount of calcium oxide is added to the first solution to create a second solution;
d) agitating the second solution to increase a rate or amount of dissociation of the calcium oxide; and
e) measuring the pH of the second solution to determine that it has a pH of about 12.5.
f) diluting the second solution to a pH of about 7.5 to produce a diluted resultant mixture; and
g) administering an alkalinity increasing amount of the diluted resultant mixture to the mammalian subject.

7. The method of claim 6, wherein the diluted resultant mixture has a conductivity of about 60 μS/cm.

8. The method of claim 6, wherein the alkalinity increasing amount comprises between about 1 to about 2 liters of the composition per day.

9. The method of claim 6, wherein the alkalinity increasing effective amount comprises about 0.5 liters every eight hours.

10. An alkalizing composition taken during exercise to improve muscle performance, speed muscle recovery, and extend endurance during exercise wherein a 0.5 liter serving comprises:
a) 0.5 liters of a dilution to a pH of 7.5 of a first solution prepared by adding between about 500 mg to about 600 mg of calcium oxide to a liter of water and agitating the first solution to increase a rate or amount of dissociation of the calcium oxide; and
b) flavoring.

11. The alkalizing composition of claim 10, wherein the 0.5 liter serving has a conductivity of about 60 μS/cm.

12. The alkalizing composition of claim 10, wherein the flavoring is at least one fruit or vegetable juice.

13. The alkalizing composition of claim 12, wherein the fruit juice is apple, cranberry, pear, peach, plum, apricot, nectarine, grape, guava, cherry, currant, raspberry, gooseberry, elderberry, blackberry, blueberry, strawberry, lemon, lime, mandarin, orange, tomato, lettuce, dandelion, rhubarb, pineapple, coconut, pomegranate, kiwi, mango, papaya, banana, watermelon, passion fruit, tangerine, or cantaloupe juice.

14. The alkalizing composition of claim 12, wherein the vegetable juice is celery, spinach, cabbage, watercress, carrot, beet, spirulina, sweet potato, kale, romaine, collard greens, endive, escarole, bok Choy, fennel, parsley, wheat grass, or cucumber.

15. The alkalizing composition of claim 10, further comprising at least one herbal supplement.

16. The alkalizing composition of claim 15, wherein the at least one herbal supplement is passion flower, horny goat weed, skullcap, milk thistle, Echinacea, dandelion leaf, St. John's wort, green tea, black tea, chamomile or peppermint, or an extract thereof.

17. The alkalizing composition of claim 10, further comprising plants or plant extracts. wherein the plants are guarana seeds, acerola berries, coconut water, yerba mate, acai berry, ginseng root, panax ginseng root, ginkgo biloba, white willow bark, acacia, ashwagandha, chokeberry, elderberry, cranberry, maqui berry, blueberry, pomegranate, rooibos, goji berry, elder berry, valerian, seabuckthorn, yumberry, blackberry, astragalus, damiana, and ginger.

18. The alkalizing composition of claim 10, further comprising at least one antioxidant.

19. The alkalizing composition of claim 18, wherein the at least one antioxidant is beta-carotene, lutein, lycopene, carotenes, vitamin A, vitamin C, and vitamin E, zinc, melatonin and carotenes, coenzyme Q, or erythorbic acid.

20. The alkalizing composition of claim 18, wherein the antioxidant is in a plant product.

21. The alkalizing composition of claim 20, wherein the plant product is apricot, acai fruit, apple, blueberry, blackberry, black currant, carrots, cherry, chokeberry, cranberry, elderberry, green tea, goji berry, grape seed, mangosteen, maqui berry, milk thistle, pomegranate seed, prune, raspberry, red grape, rooibos, rosehips, strawberry, seabuckthorn, white grape, whole grape, yumberry or acerola fruit.

22. The alkalizing composition of claim 10, further comprising at least one vitamin, mineral or nutritional supplement.

23. The alkalizing composition of claim 22, wherein the vitamin, mineral, or nutritional supplement is vitamin B complex, folic acid, niacin, niacinamide, pantothenic acid, pyridoxine HCl, vitamin B2, folate, biotin, vitamin C, vitamin D, vitamin D3, vitamin E, vitamin K, cyanocobalamin, inositol, thiamine, thiamine mononitrate, calcium pantothenate, mixed tocophyerols, d-alpha tocopheryl acetate, magnesium, calcium, calcium carbonate, calcium chelate, calcium di-phosphate, calcium phosphate, iron, magnesium carbonate, magnesium citrate, magnesium oxide, magnesium phosphate, manganese chelate, manganese sulfate, potassium, potassium chelate, potassium chloride, sodium, zinc, vanadyl sulphate, chromium, chromium chloride, chromium picolinate, or chromium polynicotinate.

24. The alkalizing composition of claim 23, wherein the composition comprises between 10% and 250% of the recommended daily allowance of vitamin B complex.

25. The alkalizing composition of claim 23, wherein the composition comprises between 10% and 250% of the recommended daily allowance of vitamin B₃.

26. The alkalizing composition of claim 10, further comprising a mixture of amino acids, amino acid precursors, and amino acid derivatives.

27. The alkalizing composition of claim 26, wherein the amino acids, amino acid derivatives, and amino acid precursors are 5-HTP, arginine, beta alanine, carnitine fumarate, citrulline malate, glutamine peptide, glycine, 1-alanine, 1-arginine, 1-arginine hydrochloride, 1-histidine, 1-methionine, 1-lysine HCL, L-phenylalanine, leucine ethyl ester, 1-glutamine, 1-isoleucine, 1-theanine, 1-tyrosine, phenylalanine, taurine, tri-methyl glycine, tryptophan, tyrosine, 1-carnitine, 1-carnosine, glutamine alpha ketoglutarate, and alpha-L-polylactate.

28. The alkalizing composition of claim 10, further comprising one or more stimulants.

29. The alkalizing composition of claim 28, wherein the one or more stimulants are caffeine, citicoline, d-glucuronolactone, guarana extract, ginseng, concentrated green tea, green coffee beans, glucuronolactone, guarana, panax ginseng, panax quinquefolius, Siberian ginseng, and theobromine.

30. The alkalizing composition of claim 10, further comprising an extract from at least one flavonoid containing compound.

31. The alkalizing composition of claim 30, wherein the flavonoid containing compound is chamomile leaves, cocoa powder, red grape, brewed black tea, brewed white tea, and anthocyanins.

32. The alkalizing composition of claim 10, further comprising at least one herbal sedative.

33. The alkalizing composition of claim 32, wherein the at least one herbal sedative is lavender, lemon balm, lemongrass, linden, oatstraw, St. John's wart, valerian root, Kava Kava, hops or passion flower.

34. The alkalizing composition of claim 10, further comprising at least one sweetener.

35. The alkalizing composition of claim 34, wherein the sweetener is acesulfame potassium, aspartame, cane sugar, corn syrup, crystalline fructose, dextrose, D-ribose, fructose, glucose, glucose-fructose syrup, high fructose corn syrup, high fructose liquid sugar, honey, maltodextrin, sorbitol, stevia, sucralose, sucrose, sugar, trehalose, truvia or xylitol.

36. The alkalizing composition of claim 10, further comprising at least one electrolyte.

37. The alkalizing composition of claim 36, wherein the at least one electrolyte is sodium chloride, sodium acetate, acidic sodium citrate, acidic sodium phosphate, sodium chloride, sodium bicarbonate, sodium bromide, sodium citrate, sodium lactate, sodium molybdate, sodium phosphate, anhydrous sodium sulphate, sodium sulphate, sodium tartrate, sodium benzoate, sodium selenite, potassium chloride, potassium acetate, potassium bicarbonate, potassium bromide, potassic citrate, potassium-D-gluconate, monobasic potassium phosphate, potassium tartrate, potassium sorbate, potassium iodide, magnesium carbonate, magnesium citrate, magnesium oxide, magnesium phosphate, calcium chloride, calcium carbonate, calcium chelate, calcium di-phosphate, calcium lactate, or calcium phosphate tribasic.

38. The alkalizing composition of claim 10, further comprising at least one adaptogen.

39. The alkalizing composition of claim 38, wherein the adaptogen is an extract from ashwagandha, eleutherococcus senticosus, reishi, astragalus, licorice root, panax quinquefolius, panax ginseng, or schisandra berries.

40. The alkalizing composition of claim 10, further comprising at least one anti-inflammatory agent.

41. The alkalizing composition of claim 40, wherein the anti-inflammatory agent is a plant extract from a plant selected from maqui berry, milk thistle, skull cap, red raspberry, red sour cherry, green tea and hops.

42. The alkalizing composition of claim 10, further comprising at least one anti-nausea agent.

43. The alkalizing composition of claim 42, wherein the at least one anti-nausea agent is an extract from peppermint, ginger, or chamomile.

44. The alkalizing composition of claim 10, further comprising at least one energy booster.

45. The alkalizing composition of claim 44, wherein the at least one energy booster is creatine ethyl ester, creatine monohydrate, magnesium creatine chelate, creatine hydrochloride, creatine nitrate, creatine monohydrate, or royal jelly.

46. The alkalizing composition of claim 10, further comprising at least one source of flavonoids.

47. The alkalizing composition of claim 46, wherein the at least one source of flavonoids is chamomile extract, cocoa powder, red grape, black tea, white tea, ginkgo biloba, berries, parsley, and green tea.

48. A method of preventing acidosis resulting from strenuous bodily activity comprising administering a beverage comprising effective amounts of alkaline water immediately prior to strenuous bodily activity, wherein the alkaline water is a 0.5 liters of a dilution to a pH of 7.5 of a first solution prepared by adding between about 500 mg to about 600 mg of calcium oxide to a liter of water and agitating the first solution to increase a rate or amount of dissociation of the calcium oxide.

49. The method of claim 48, wherein the beverage further comprises a flavoring.

50. The method of claim 49, wherein the flavoring is at least one fruit or vegetable juice.

51. The method of claim 49, wherein the fruit juice is apple, cranberry, pear, peach, plum, apricot, nectarine, grape, guava, cherry, currant, raspberry, gooseberry, elderberry, blackberry, blueberry, strawberry, lemon, lime, mandarin, orange, tomato, lettuce, dandelion, rhubarb, pineapple, coconut, pomegranate, kiwi, mango, papaya, banana, watermelon, passion fruit, tangerine, or cantaloupe juice.

52. The method of claim 49, wherein the vegetable juice is celery, spinach, cabbage, watercress, carrot, beet, spirulina, sweet potato, kale, romaine, collard greens, endive, escarole, bok choy, fennel, parsley, wheat grass, or cucumber.

53. The method of claim 48, wherein the beverage further comprises at least one herbal supplement.

54. The method of claim 53, wherein the at least one herbal supplement is passion flower, horny goat weed, skullcap, milk thistle, Echinacea, dandelion leaf, St. John's wort. green tea, black tea, chamomile or peppermint, or an extract thereof.

55. The method of claim 48, wherein the beverage further comprises plants or plant extracts, wherein the plants are guarana seeds, acerola berries, coconut water, yerba mate, acai berry, ginseng root, panax ginseng root, ginkgo biloba, white willow bark, acacia, ashwagandha, chokeberry, elderberry, cranberry, maqui berry, blueberry, pomegranate, rooibos, goji berry, elder berry, valerian, seabuckthorn, yumberry, blackberry, astragalus, damiana, and ginger.

56. The method of claim 48, wherein the beverage further comprises at least one antioxidant.

57. The method of claim 56, wherein the antioxidant is beta-carotene, lutein, lycopene, carotenes, vitamin A, vitamin C, and vitamin E, zinc, melatonin and carotenes, coenzyme Q, or erythorbic acid.

58. The method of claim 56, wherein the antioxidant is in a plant product.

59. The method of claim 58, wherein the plant product is apricot, acai fruit, apple, blueberry, blackberry, black currant, carrots, cherry, chokeberry, cranberry, elderberry, green tea, goji berry, grape seed, mangosteen, maqui berry, milk thistle, pomegranate seed, prune, raspberry, red grape, rooibos, rosehips, strawberry, seabuckthorn, white grape, whole grape, yumberry or acerola fruit.

60. The method of claim 48, wherein the beverage further comprises at least one vitamin, mineral or nutritional supplement.

61. The method of claim 60, wherein the vitamin, mineral, or nutritional supplement is vitamin B complex, folic acid, niacin, niacinamide, pantothenic acid, pyridoxine hydrochloride, vitamin B2, folate, biotin, vitamin C, vitamin D, vitamin D3, vitamin E, vitamin K, cyanocobalamin, inositol, thiamine, thiamine mononitrate, calcium pantothenate, mixed tocophyerols, d-alpha tocopheryl acetate, magnesium, calcium, calcium carbonate, calcium chelate, calcium di-phosphate, calcium phosphate, iron, magnesium carbonate, magnesium citrate, magnesium oxide, magnesium phosphate, manganese chelate, manganese sulfate, potassium, potassium chelate, potassium chloride, sodium, zinc, vanadyl sulphate, chromium, chromium chloride, chromium picolinate, or chromium polynicotinate.

62. The method of claim 48, wherein the beverage further comprises a mixture of amino acids, amino acid precursors, and amino acid derivatives.

63. The method of claim 62, wherein the amino acids, amino acid derivatives, and amino acid precursors are 5-HTP, arginine, beta alanine, carnitine fumarate, citrulline malate, glutamine peptide, glycine, 1-alanine, 1-arginine, 1-arginine hydrochloride, 1-histidine, 1-methionine, 1-lysine HCL, L-phenylalanine, leucine ethyl ester, 1-glutamine, 1-isoleucine, 1-theanine, 1-tyrosine, phenylalanine, taurine, tri-methyl glycine, tryptophan, tyrosine, 1-carnitine, 1-carnosine, glutamine alpha ketoglutarate, and alpha-L-polylactate.

64. The method of claim 48, wherein the beverage further comprises one or more stimulants.

65. The method of claim 64, wherein the one or more stimulants are caffeine, citicoline, d-glucuronolactone, guarana extract, ginseng, concentrated green tea, green coffee beans, glucuronolactone, guarana, panax ginseng, panax quinquefolius, Siberian ginseng, and theobromine.

66. The method of claim 48, wherein the beverage further comprises an extract from at least one flavonoid containing compound.

67. The method of claim 66, wherein the flavonoid containing compound is chamomile leaves, cocoa powder, red grape, brewed black tea, brewed white tea, and anthocyanins.

68. The method of claim 48, wherein the beverage further comprises at least one herbal sedative.

69. The method of claim 68, wherein the at least one herbal sedative is lavender, lemon balm, lemongrass, linden, oatstraw, St. John's wart, valerian root, Kava Kava, hops or passion flower.

70. The method of claim 48, wherein the beverage further comprises at least one sweetener.

71. The method of claim 70, wherein the sweetener is acesulfame potassium, aspartame, cane sugar, corn syrup, crystalline fructose, dextrose, D-ribose, fructose, glucose, glucose-fructose syrup, high fructose corn syrup, high fructose liquid sugar, honey, maltodextrin, sorbitol, stevia, sucralose, sucrose, sugar, trehalose, truvia or xylitol.

72. The method of claim 48, wherein the beverage further comprises at least one electrolyte.

73. The method of claim 72, wherein the at least one electrolyte is sodium chloride, sodium acetate, acidic sodium citrate, acidic sodium phosphate, sodium chloride, sodium bicarbonate, sodium bromide, sodium citrate, sodium lactate, sodium molybdate, sodium phosphate, anhydrous sodium sulphate, sodium sulphate, sodium tartrate, sodium benzoate, sodium selenite, potassium chloride, potassium acetate, potassium bicarbonate, potassium bromide, potassium citrate, potassium-D-gluconate, monobasic potassium phosphate, potassium tartrate, potassium sorbate, potassium iodide, magnesium carbonate, magnesium citrate, magnesium oxide, magnesium phosphate, calcium chloride, calcium carbonate, calcium chelate, calcium di-phosphate, calcium lactate, or calcium phosphate tribasic.

74. The method of claim 48, wherein the beverage further comprises at least one adaptogen.

75. The method of claim 74, wherein the adaptogen is an extract from ashwagandha., eleutherococcus senticosus, reishi, astragalus, licorice root, panax quinquefolius, panax ginseng, or schisandra berries.

76. The method of claim 48, wherein the beverage further comprises at least one anti-inflammatory agent.

77. The method of claim 76, wherein the anti-inflammatory agent is a plant extract from a plant selected from maqui berry, milk thistle, skull cap, red raspberry, red sour cherry, green tea and hops.

78. The method of claim 48, wherein the beverage further comprises at least one anti-nausea agent.

79. The method of claim 78, wherein the at least one anti-nausea agent is an extract from peppermint, ginger or chamomile.

80. The method of claim 48, wherein the beverage further comprises at least one energy booster.

81. The method of claim 80, wherein the at least one energy booster is creatine ethyl ester, creatine monohydrate, magnesium creatine chelate, creatine hydrochloride, creatine nitrate, creatine monohydrate or royal jelly.

82. The method of claim 48, wherein the beverage further comprises at least one source of flavonoids.

83. The method of claim 82, wherein the at least one source of flavonoids is chamomile extract, cocoa powder, red grape, black tea, white tea, ginkgo biloba, berries, parsley, and green tea.

84. A method of maintaining proper physiological pH levels in an individual with or at risk for acidosis comprising administering a beverage comprising the daily consumption of effective amounts of alkaline water, wherein the alkaline water is a 0.5 liters of a dilution to a pH of 7.5 of a first solution prepared by adding between about 500 mg to about 600 mg of calcium oxide to a liter of water and agitating the first solution to increase a rate or amount of dissociation of the calcium oxide.

85. The method of claim 84, wherein the effective amount is 2 L per day.
